(19) United States
(12) Patent Application Publication
(10) Pub. No.: US 2004/0220187 A1
(43) Pub. Date: Nov. 4, 2004

(54) Compositions of a cyclooxygenase-2 selective inhibitor and a sodium ion channel blocker for the treatment of pain, inflammation or inflammation mediated disorders

(75) Inventors: Diane T. Stephenson, Groton, CT (US); Duncan P. Taylor, Bridgewater, MI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/828,683

(22) Filed: Apr. 21, 2004

Related U.S. Application Data
(60) Provisional application No. 60/464,775, filed on Apr. 23, 2003.
(60) Provisional application No. 60/464,609, filed on Apr. 22, 2003.

(51) Int. Cl. A61K 31/50; A61K 31/415

(52) U.S. Cl. 514/247; 514/406; 514/374; 514/561

(57) ABSTRACT

The present invention provides compositions and methods for the treatment of pain, inflammation or inflammation-mediated disorders in a subject. More particularly, the invention provides a combination therapy for the treatment of pain, inflammation or inflammation mediated disorders comprising the administration to a subject of a sodium ion channel blocker in combination with a cyclooxygenase-2 selective inhibitor.

### CROSS REFERENCE TO RELATED APPLICATION

This application claims priority from the following Provisional Applications: Serial No. 60/464,775 filed on Apr. 23, 2003, and Serial No. 60/464,609 filed on Apr. 22, 2003, all of which are hereby incorporated by reference in their entirety.

### FIELD OF THE INVENTION

The present invention provides methods and compositions related to the treatment of pain, inflammation or inflammation mediated disorders. More particularly, the invention is directed toward a combination therapy for the treatment of pain, inflammation or inflammation mediated disorders comprising the administration to a subject of a sodium ion channel blocker in combination with a cyclooxygenase-2 selective inhibitor.

### BACKGROUND OF THE INVENTION

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. Pain sensation is complex and variable. Often experiences considered painful by one subject may not be equally painful to another and may vary in the same subject depending on the circumstances presented. This range of sensations, as well as the variation in perception of pain by different individuals, renders a precise definition of pain difficult, however, many individuals suffer with severe and continuous pain.

Pain can be caused by the stimulation of nociceptive receptors and transmitted over intact neural pathways, in which case the pain is termed “nociceptive” pain. Generally speaking, there are two different types of nociceptive stimuli that are intense enough to be perceived as pain. One type, somatic pain, consists of an intense, localized, sharp or stinging sensation. Somatic pain is mediated by fast-conducting, lightly myelinated A-delta fibers that have a high threshold (i.e. require a strong mechanical stimulus to sense pain) and enter into the spinal cord through the dorsal horn of the central nervous system where they terminate in the spinal cord.

The second type of pain, sometimes referred to as visceral pain, is characterized as a diffuse, dull, aching or burning sensation. Visceral pain is mediated largely by unmyelinated, slower-conducting C-fibers that are polymodal (i.e., mediate mechanical, thermal, or chemical stimuli). C-fibers also enter the spinal cord through the dorsal horn of the central nervous system where they terminate in the spinal cord. Both somatic and visceral pain can be sensed centrally and peripherally within the human body and may be either acute or chronic.

A number of analgesics reduce both central and peripheral sensitization through interaction with the various pain-based receptors within the human body. For example, morphine and most other opioid analgesics elicit an inhibitory neuronal effect within central nervous and gastrointestinal (GI) systems by interacting with areas of the brain receiving input from the spinal pain-transmitting pathways containing opioid receptors. By suppressing neuronal activity at these receptor points, opioid narcotics produce analgesia and control the pain threshold within a human patient.

Opioid narcotics, however, have several negative side effects that severely limit their therapeutic value. These side effects include drowsiness, lethargy, difficulty in being mobile, respiratory depression, excessive central nervous system depression, weakness in the extremities, and dizziness. In addition, patients being treated with opioids also may develop tolerance to the agent, requiring higher doses, or addition of other opioids to the pain treatment regimen. The larger effective dosage may in turn lead to the development of physical and psychological addiction. Further, other typical side effects of opioid analgesics include miosis, or constriction of the pupils, nausea, vomiting, prolongation of stomach emptying time, and decreased propulsive contractions of the small intestine.

As an alternative to opioid analgesics, a number of non-narcotic based drugs may be utilized to treat mild to moderate pain. In a recent study, sodium channel blockers were shown to be effective analgesics in the treatment of pain (Laird J. M., et al., (2001) Br J Pharmacol Dec; 134(8): 1742-8). Neuropathic pain is thought to result from sustained firing of sensory neurons. One study demonstrated the introduction of a sodium channel blocker resulted in radically altered firing behavior of an unmodified Hodgkin-Huxley axon (Elliott, J. (1997) Brain Research; 754:221-26). Generally speaking, non-narcotic drugs can be given over longer periods of time compared to opioid analgesics because of their lower central nervous system and respiratory depressive effects. Examples of non-narcotic drugs employed to treat pain include acetylsalicylic acid (aspirin), centrally acting alpha antiadrenergic agents, diflusinal, salsalate, acetaminophen, and nonsteroidal anti-inflammatory agents such as ibuprofen, naproxen, and fenoprofen. These agents all generally relieve pain through prostaglandin synthesis inhibition resulting in a decrease in pain receptor stimulation.

Non-narcotic drugs also have several negative side effects that severely limit their therapeutic value. Aspirin, for example, has been shown through epidemiological data to be a factor in the occurrence of Reye's syndrome. In addition, salicylates have been shown to cause gastrointestinal upset, gastrointestinal hemorrhage, and anti-platelet effects. Acetaminophen has been linked to liver damage, kidney damage, and hematological effects such as hemolytic anemia, neutropenia, and leukopenia. Moreover, nonsteroidal anti-inflammatory agents also exhibit numerous negative side effects as well, ranging from gastrointestinal distress, gastrointestinal hemorrhage, and kidney damage when administered at a therapeutically effective dosage for the treatment of pain.

### SUMMARY OF THE INVENTION

Among the several aspects of the invention is provided a method for the treatment of pain, inflammation or inflammation-mediated disorders in a subject. The method comprises administering to the subject a cyclooxygenase-2 selective inhibitor or a pharmaceutically acceptable salt or a prodrug thereof in combination with a sodium ion channel blocker or pharmaceutically acceptable salt or prodrug thereof.

In one embodiment, the cyclooxygenase-2 selective inhibitor is a member of the chromene class of compounds. For example, the chromene compound may be a compound of the formula:

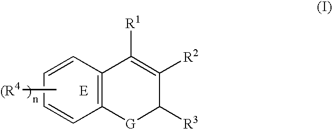

wherein:

n is an integer which is 0, 1, 2, 3 or 4;

G is O, S or NR^{a};

R^{a} is alkyl;

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical;

or prodrug thereof.

In another embodiment, the cyclooxygenase-2 selective inhibitor or a pharmaceutically acceptable salt or a prodrug thereof comprises a compound of the formula:

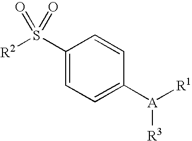

wherein

A is selected from the group consisting of partially unsaturated or unsaturated heterocyclyl and partially unsaturated or unsaturated carbocyclic rings;

R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of methyl or amino; and

R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In one embodiment, the sodium ion channel blocker is a member of the type IA antiarrythmic class of compounds.

In a further embodiment, the sodium ion channel blocker is a member of the type IB antiarrythmic class of compounds.

In still a further embodiment, the sodium ion channel blocker is a member of the type IC antiarrythmic class of compounds.

In yet another embodiment, the sodium ion channel blocker is lubeluzole or a pharmaceutically acceptable salt or prodrug thereof.

Other aspects of the invention are described in more detail below.

Abbreviations and Definitions

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH2.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” Is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO2H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to inhibit cyclooxygenase-2 without significant inhibition of cyclooxygenase-1. Typically, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more typically, of at least 100. Even more typically, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2—) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “subject” for purposes of treatment includes any human or animal subject who is in need of such treatment. The subject can be a domestic livestock species, a laboratory animal species, a zoo animal or a companion animal. In one embodiment, the subject is a mammal. In another embodiment, the mammal is a human being.

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of each agent (i.e. the amount of cyclooxygenase-2 selective inhibitor and the amount of sodium ion channel blocker) which will achieve the goal of improvement in disorder severity and the frequency of incidence over no treatment or treatment of each agent by itself.

### DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of a sodium ion channel blocker. The combination therapy may be used to treat a pain, inflammation or an inflammation mediated disorder. When administered as part of a combination therapy, the COX-2 selective inhibitor together with the sodium ion channel blocker provide enhanced treatment options as compared to administration of either the sodium ion channel blocker or the COX-2 selective inhibitor alone.

Cyclooxygenase-2 Selective Inhibitors

A number of suitable cyclooxygenase-2 selective inhibitors or an isomer, a pharmaceutically acceptable salt, ester, or prodrug thereof, may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

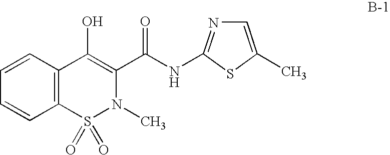

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

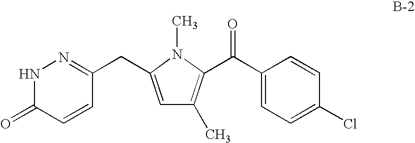

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having Formula I shown below and possessing, by way of example and not limitation, the structures disclosed in Table 1x. Furthermore, benzopyran cyclooxygenase-2 selective inhibitors useful in the practice of the present methods are described in U.S. Pat. No. 6,034,256 and 6,077,850 herein incorporated by reference in their entirety.

In another embodiment, the cyclooxygenase-2 selective inhibitor is a chromene compound represented by Formula/or an isomer, a pharmaceutically acceptable salt, ester, or prodrug thereof:

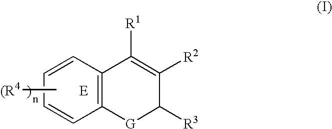

wherein:

n is an integer which is 0, 1, 2, 3 or 4;

G is O, S or NR^{a};

R^{a} is alkyl

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof, wherein:

n is an integer which is 0, 1, 2, 3 or 4;

G is O, S or NR^{a};

R¹ is H;

R^{a} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I), or an isomer, a pharmaceutically acceptable salt, ester, or prodrug thereof, wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

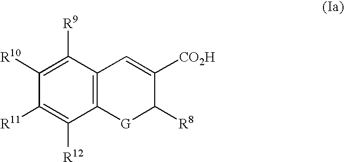

The cyclooxygenase-2 selective inhibitor used in connection with the method(s) of the present invention can also be a compound of having the structure of Formula (Ia) or an isomer, a pharmaceutically acceptable salt, ester, or prodrug thereof wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1×below.

TABLE 1x
Examples of Chromene Cyclooxygenase-2
Selective Inhibitors as Embodiments

| Compound | |
|---|---|
| Number | Structural Formula |
| B-3 | 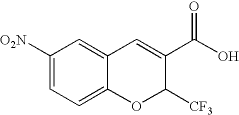 |
| | 6-Nitro-2-trifluoromethyl-2H-1- |
| | benzopyran-3-carboxylic acid |
| B-4 | 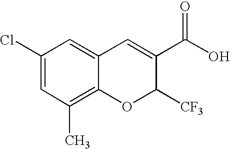 |
| | 6-Chloro-8-methyl-2-trifluoromethyl- |
| | 2H-1-benzopyran-3-carboxylic acid |
| B-5 | 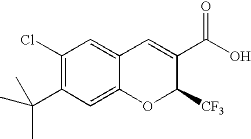 |
| | ((S)-6-Chloro-7-(1,1-dimethylethyl)-2- |
| | (triflouromethyl-2H-1-benzopyran-3- |
| | carboxylic acid |
| B-6 | 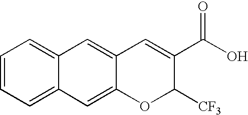 |
| | 2-Trifluoromethyl-2H-naphtho[2,3-b] |
| | pyran-3-carboxylic acid |
| B-7 | 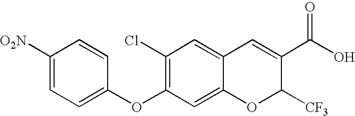 |
| | 6-Chloro-7-(4-nitrophenoxy)-2-(trifluoromethyl)- |
| | benzopyran-3-carboxylic acid |
| B-8 | 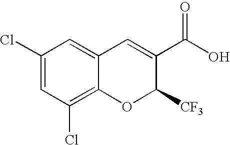 |
| | ((S)-6,8-Dichloro-2-(trifluoromethyl)- |
| | 2H-1-benzopyran-3-carboxylic acid |
| B-9 | 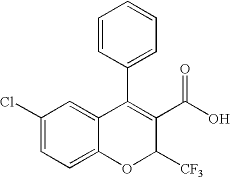 |
| | 6-Chloro-2-(trifluoromethyl)-4-phenyl-2H- |
| | 1-benzopyran-3-carboxylic acid |
| B-10 | 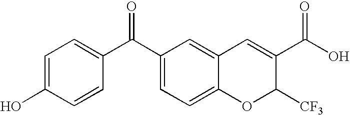 |
| | 6-(4-Hydroxybenzoyl)-2-(trifluoromethyl)- |
| | 2H-1-benzopyran-3-carboxylic acid |
| B-11 | 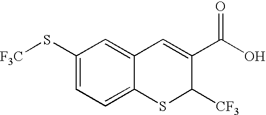 |
| | 2-(Trifluoromethyl)-6-[(trifluoromethyl)thio]- |
| | 2H-1-benzothiopyran-3-carboxylic acid |
| B-12 | 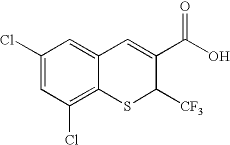 |
| | 6,8-Dichloro-2-trifluoromethyl-2H-1- |
| | benzothiopyran-3-carboxylic acid |
| B-13 | 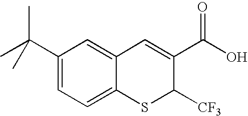 |
| | 6-(1,1-Dimethylethyl)-2-(trifluoromethyl)- |
| | 2H-1-benzothiopyran-3-carboxylic acid |
| B-14 | 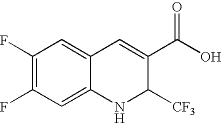 |
| | 6,7-Difluoro-1,2-dihydro-2-(trifluoromethyl)- |
| | 3-quinolinecarboxylic acid |
| B-15 | 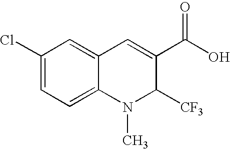 |
| | 6-Chloro-1,2-dihydro-1-methyl-2-(trifluoromethyl)- |
| | 3-quinolinecarboxylic acid |
| B-16 | 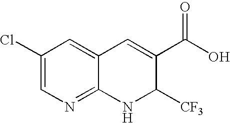 |
| | 6-Chloro-2-(trifluoromethyl)-1,2-dihydro |
| | [1,8]naphthyridine-3-carboxylic acid |
| B-17 | 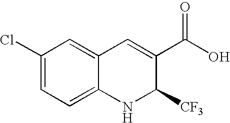 |
| | ((S)-6-Chloro-1,2-dihydro-2-(trifluoromethyl)- |
| | 3-quinolinecarboxylic acid |

In a further embodiment, the cyclooxygenase-2 selective inhibitor is selected from the class of tricyclic cyclooxygenase-2 selective inhibitors represented by the general structure of Formula I: or an isomer, a pharmaceutically acceptable salt, ester, or prodrug thereof wherein:

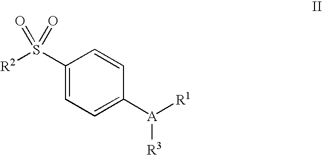

A is selected from the group consisting of partially unsaturated or unsaturated heterocyclyl and partially unsaturated or unsaturated carbocyclic rings;

R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of methyl or amino; and

R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In another embodiment, the cyclooxygenase-2 selective inhibitor represented by the above Formula II is selected from the group of compounds illustrated in Table 2x, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), tilmacoxib (JTE-522; B-23; CAS No. 180200-68-4).

TABLE 2x
Examples of Tricyclic Cyclooxygenase-2
Selective Inhibitors as Embodiments

| Compound | |
|---|---|
| Number | Structural Formula |
| B-18 | 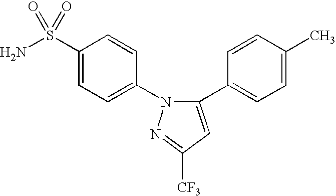 |
| B-19 | 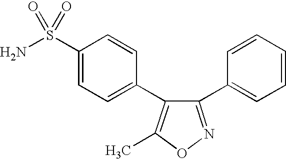 |
| B-20 | 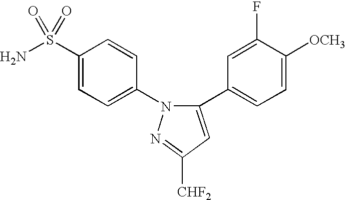 |
| B-21 | 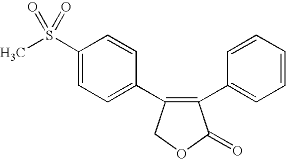 |
| B-22 | 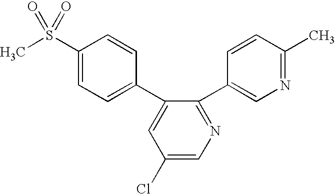 |
| B-23 | 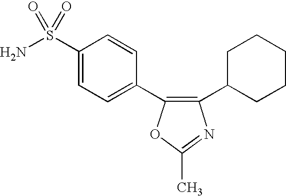 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

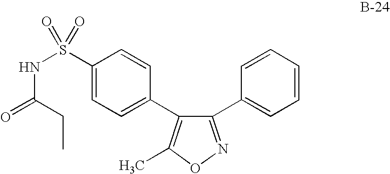

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

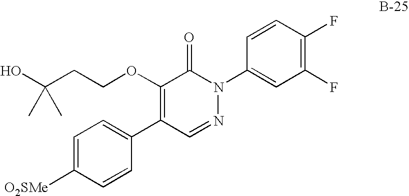

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

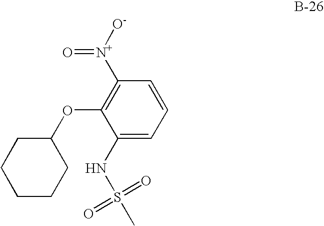

In yet a further embodiment, the cyclooxygenase-2 selective inhibitor used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug thereof:

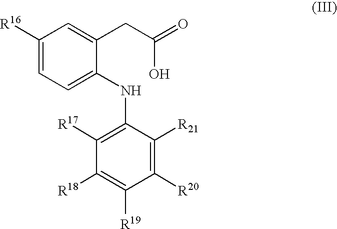

wherein:

R¹⁶ is methyl or ethyl;

R¹⁷ is chloro or fluoro;

R¹⁸ is hydrogen or fluoro;

R⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-211) and that has the structure shown in Formula (III) or an isomer, a pharmaceutically acceptable salt, ester, or prodrug thereof wherein:

R¹⁶ is ethyl;

R¹⁷ and R¹⁹ are chloro;

R¹⁸ and R²⁰ are hydrogen; and

R²¹ is methyl.

In yet another embodiment, the cyclooxygenase-2 selective inhibitor is represented by Formula (IV) or an isomer, a pharmaceutically acceptable salt, ester, or prodrug thereof:

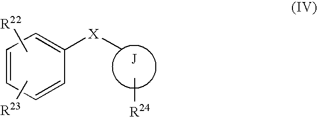

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V) or an isomer, a pharmaceutically acceptable salt, ester, or prodrug thereof:

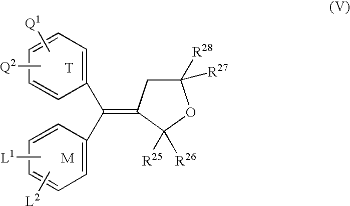

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0,1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are 0; or,

R²⁷ and R²⁸ are 0; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In another embodiment, the compounds N-(2-cyclohexyloxynitrophenyl)methane sulfonamide, and (E)-4-[(4-methylphenyl)(tetrahydro-2-oxo-3-furanylidene) methyl]benzenesulfonamide or an isomer, a pharmaceutically acceptable salt, ester, or prodrug thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3x below, include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl] benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)₄-(4-fluorophenyl)-5-(4-methylsulfonylphenyl)thiazole (B-112);

5-(4-fluorophenyl)₄-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)₄-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)₄-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]4-methyl-1 H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)₄-(trifluoromethyl)-1H-imidazol-1-yl]benzenesulfonamide (B-143);

2-(3-fluoro-5-methylphenyl)-1-[4-(methylsulfonyl)phenyl]4-(trifluoromethyl)-1H-imidazole (B-144);

4-[2-(3-fluoro-5-methylphenyl)₄-(trifluoromethyl)-1H-imidazol-1-yl]benzenesulfonamide (B-145);

2-(3-methylphenyl)-1-[4-(methylsulfonyl)phenyl]4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)₄-trifluoromethyl-1H-imidazole (B-148);

4-[2-(3-chlorophenyl)₄-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)₄-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)₄-(4-methylsulfonylphenyl)-2-trifluoromethyl-1 H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)₄-[4-(methylsulfonyl)phenyl]-6-(trifluoromethyl)pyridine (B-162);

5-(4-fluorophenyl)₄-[4-(methylsulfonyl)phenyl]-2-(2-propynyloxy)-6-(trifluoromethyl)pyridine (B-163);

2-bromo-5-(4-fluorophenyl)₄-[4-(methylsulfonyl)phenyl]-6-(trifluoromethyl)pyridine (B-164);

4-[2-(3-chloro-4-methoxyphenyl)4,5-difluorophenyl]benzenesulfonamide (B-165);

1-(4-fluorophenyl]-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]4-(methylsulfonyl)benzene (B-176);

1-[2-(4-methylthiophenyl)cyclopenten-1-yl]4-(methylsulfonyl)benzene (B-177);

1-[2-(4-fluorophenyl)4,4-dimethylcyclopenten-1-yl]4-(methylsulfonyl)benzene (B-178);

4-[2-(4-fluorophenyl)4,4-dimethylcyclopenten-1-yl]benzenesulfonamide (B-179);

1-[2-(4-chlorophenyl)4,4-dimethylcyclopenten-1-yl]4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]4-(methylsulfonyl)benzene (B-184);

1-[2-(2,3-difluorophenyl)cyclopenten-1-yl]4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl] benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)₄-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)₄-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifuoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]4-trifluoromethyl-1H-imidazol-2-yl]pyridine (B-203);

2-methyl-5-[1-[4-(methylsulfonyl)phenyl]4-trifluoromethyl-1 H-imidazol-2-yl]pyridine (B-204);

4-[2-(5-methylpyridin-3-yl)₄-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189 (lumiracoxib; B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231);

S-2474 (B-232);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methylsulfonyl)phenyl]-3(2H)-pyridzainone;

2-trifluoromethyl-3H-naptho[2,1-b]pyran-3-carboxylic acid;

6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3x
Examples of Cyclooxygenase-2 Selective Inhibitors as Embodiments

| Compound | |
|---|---|
| Number | Structural Formula |
| B-26 | 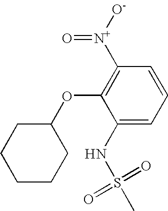 |
| | N-(2-cyclohexyloxynitrophenyl) methane |
| | sulfonamide or NS-398; |
| B-27 | 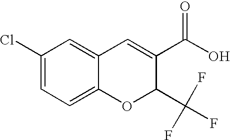 |
| | 6-chloro-2-trifluoromethyl-2H-1-benzopyran- |
| | 3-carboxylic acid; |
| B-28 | 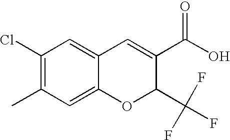 |
| | 6-chloro-7-methyl-2-trifluoromethyl-2H-1- |
| | benzopyran-3-carboxylic acid; |
| B-29 | 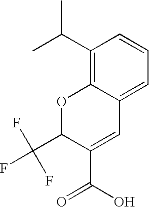 |
| | 8-(1-methylethyl)-2-trifluoromethyl-2H-1- |
| | benzopyran-3-carboxylic acid; |
| B-30 | 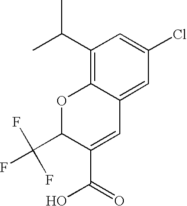 |
| | 6-chloro-8-(1-methylethyl)-2-trifluoromethyl- |
| | 2H-1-benzopyran-3-carboxylic acid; |
| B-31 | 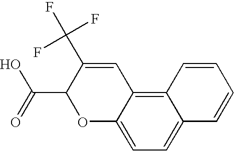 |
| | 2-trifluoromethyl-3H-naphtho[2,1-b]pyran-3- |
| | carboxylic acid; |
| B-32 | 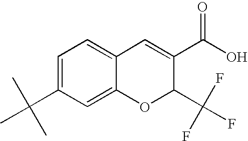 |
| | 7-(1,1-dimethylethyl)-2-trifluoromethyl-2H- |
| | 1-benzopyran-3-carboxylic acid; |
| B-33 | 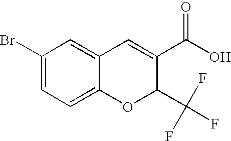 |
| | 6-bromo-2-trifluoromethyl-2H-1-benzopyran- |
| | 3-carboxylic acid; |
| B-34 | 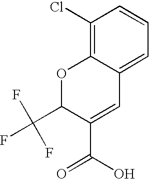 |
| | 8-chloro-2-trifluoromethyl-2H-1- |
| | benzopyran-3-carboxylic acid; |
| B-35 | 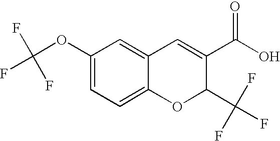 |
| | 6-trifluoromethoxy-2-trifluoromethyl- |
| | 2H-1-benzopyran-3-carboxylic acid; |
| B-36 | 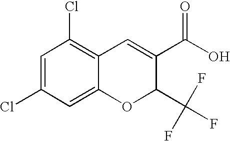 |
| | 5,7-dichloro-2-trifluoromethyl-2H-1- |
| | benzopyran-3-carboxylic acid; |
| B-37 | 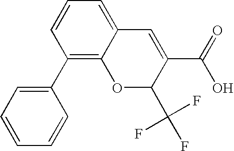 |
| | 8-phenyl-2-trifluoromethyl-2H-1- |
| | benzopyran-3-carboxylic acid; |
| B-38 | 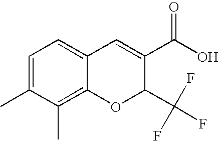 |
| | 7,8-dimethyl-2-trifluoromethyl-2H-1- |
| | benzopyran-3-carboxylic acid; |
| B-39 | 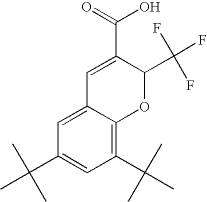 |
| | 6,8-bis(dimethylethyl)-2-trifluoromethyl- |
| | 2H-1-benzopyran-3-carboxylic acid; |
| B-40 | 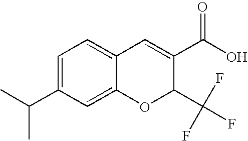 |
| | 7-(1-methylethyl)-2-trifluoromethyl-2H-1- |
| | benzopyran-3-carboxylic acid; |
| B-41 | 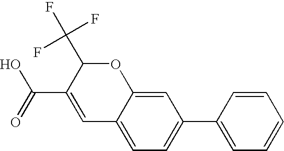 |
| | 7-phenyl-2-trifluoromethyl-2H-1-benzopyran- |
| | 3-carboxylic acid; |
| B-42 | 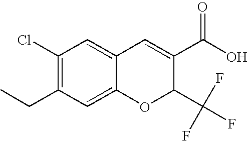 |
| | 6-chloro-7-ethyl-2-trifluoromethyl-2H-1- |
| | benzopyran-3-carboxylic acid; |
| B-43 | 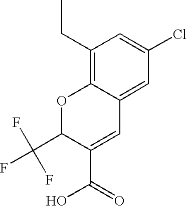 |
| | 6-chloro-8-ethyl-2-trifluoromethyl-2H-1- |
| | benzopyran-3-carboxylic acid; |
| B-44 | 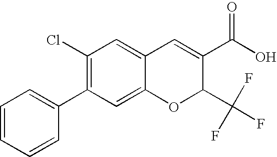 |
| | 6-chloro-7-phenyl-2-trifluoromethyl-2H-1- |
| | benzopyran-3-carboxylic acid; |
| B-45 | 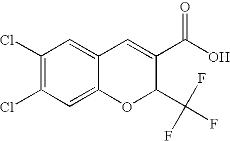 |
| | 6,7-dichloro-2-trifluoromethyl-2H-1- |
| | benzopyran-3-carboxylic acid; |
| B-46 | 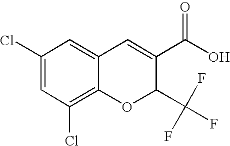 |
| | 6,8-dichloro-2-trifluoromethyl-2H-1- |
| | benzopyran-3-carboxylic acid; |
| B-47 | 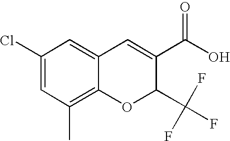 |
| | 6-chloro-8-methyl-2-trifluoromethyl-2H-1- |
| | benzopyran-3-carboxylic acid; |
| B-48 | 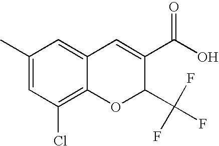 |
| | 8-chloro-6-methyl-2-trifluoromethyl-2H-1- |
| | benzopyran-3-carboxylic acid; |
| B-49 | 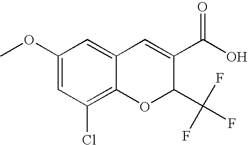 |
| | 8-chloro-6-methoxy-2-trifluoromethyl-2H-1- |
| | benzopyran-3-carboxylic acid; |
| B-50 | 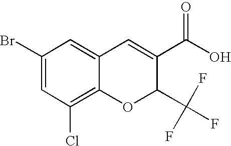 |
| | 6-bromo-8-chloro-2-trifluoromethyl-2H-1- |
| | benzopyran-3-carboxylic acid; |
| B-51 | 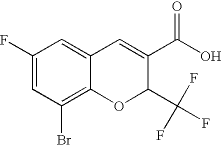 |
| | 8-bromo-6-fluoro-2-trifluoromethyl-2H-1- |
| | benzopyran-3-carboxylic acid; |
| B-52 | 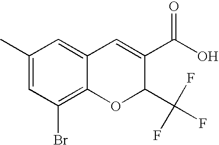 |
| | 8-bromo-6-methyl-2-trifluoromethyl-2H-1- |
| | benzopyran-3-carboxylic acid; |
| B-53 | 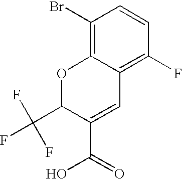 |
| | 8-bromo-5-fluoro-2-trifluoromethyl-2H-1- |
| | benzopyran-3-carboxylic acid; |
| B-54 | 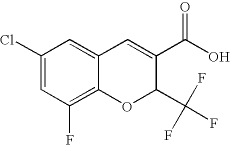 |
| | 6-chloro-8-fluoro-2-trifluoromethyl-2H-1- |
| | benzopyran-3-carboxylic acid; |
| B-55 | 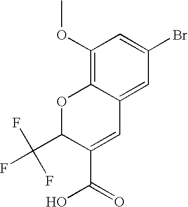 |
| | 6-bromo-8-methoxy-2-trifluoromethyl-2H-1- |
| | benzopyran-3-carboxylic acid; |
| B-56 | 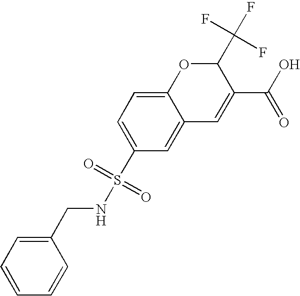 |
| | 6-[[(phenylmethyl)amino]sulfonyl]-2- |
| | trifluoromethyl-2H-1-benzopyran-3- |
| | carboxylic acid; |
| B-57 | 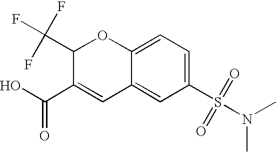 |
| | 6-[(dimethylamino)sulfonyl]-2-trifluoromethyl- |
| | 2H-1-benzopyran-3-carboxylic acid; |
| B-58 | 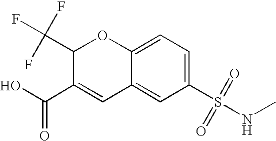 |
| | 6-[(methylamino)sulfonyl]-2-trifluoromethyl-2H- |
| | 1-benzopyran-3-carboxylic acid; |
| B-59 | 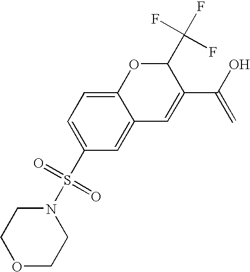 |
| | 6-[(4-morpholino)sulfonyl]-2-trifluoromethyl- |
| | 2H-1-benzopyran-3-carboxylic acid; |
| B-60 | 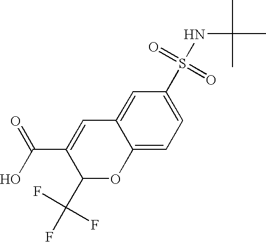 |
| | 6-[(1,1-dimethylethyl)aminosulfonyl]-2- |
| | trifluoromethyl-2H-1-benzopyran-3- |
| | carboxylic acid; |
| B-61 | 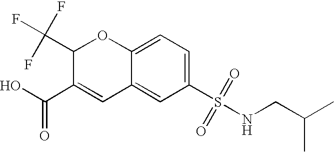 |
| | 6-[(2-methylpropyl)aminosulfonyl]-2-trifluoromethyl- |
| | 2H-1-benzopyran-3-carboxylic acid; |
| B-62 | 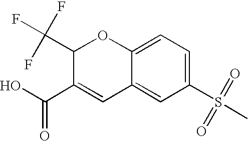 |
| | 6-methylsulfonyl-2-trifluoromethyl-2H-1-benzopyran- |
| | 3-carboxylic acid; |
| B-63 | 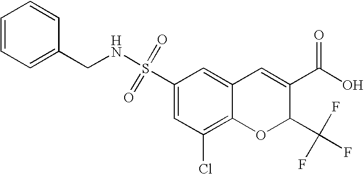 |
| | 8-chloro-6-[[(phenylmethyl)amino]sulfonyl]-2- |
| | trifluoromethyl-2H-1-benzopyran-3-carboxylic acid; |
| B-64 | 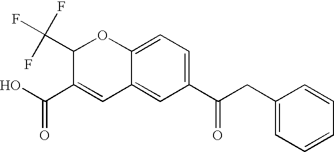 |
| | 6-phenylacetyl-2-trifluoromethyl-2H-1-benzopyran- |
| | 3-carboxylic acid; |
| B-65 | 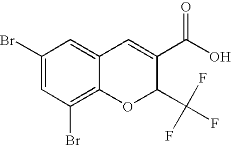 |
| | 6,8-dibromo-2-trifluoromethyl-2H-1-benzopyran- |
| | 3-carboxylic acid; |
| B-66 | 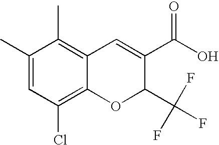 |
| | 8-chloro-5,6-dimethyl-2-trifluoromethyl-2H-1- |
| | benzopyran-3-carboxylic acid; |
| B-67 | 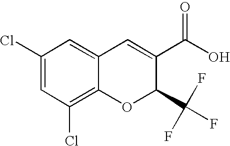 |
| | 6,8-dichloro-(S)-2-trifluoromethyl-2H-1- |
| | benzopyran-3-carboxylic acid; |
| B-68 | 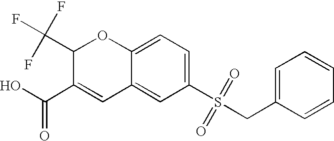 |
| | 6-benzylsulfonyl-2-trifluoromethyl-2H-1- |
| | benzopyran-3-carboxylic acid; |
| B-69 | 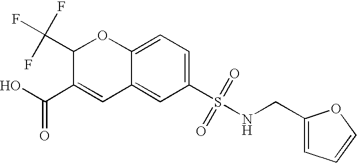 |
| | 6-[[N-(2-furylmethyl)amino]sulfonyl]-2- |
| | trifluoromethyl-2H-1-benzopyran-3-carboxylic acid; |
| B-70 | 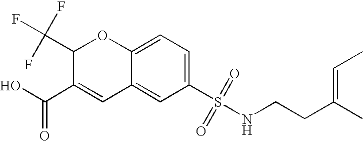 |
| | 6-[[N-(2-phenylethyl)amino]sulfonyl]-2- |
| | trifluoromethyl-2H-1-benzopy-3-carboxylic acid; |
| B-71 | 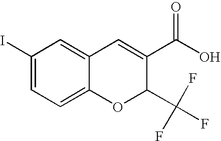 |
| | 6-iodo-2-trifluoromethyl-2H-1-benzopyran-3- |
| | carboxylic acid; |
| B-72 | 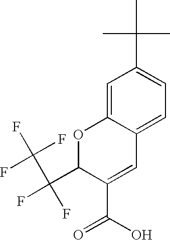 |
| | 7-(1,1-dimethylethyl)-2-pentafluoroethyl-2H- |
| | 1-benzopyran-3-carboxylic acid; |
| B-73 | 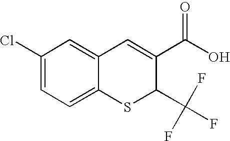 |
| | 6-chloro-2-trifluoromethyl-2H-1-benzothiopyran- |
| | 3-carboxylic acid; |
| B-74 | 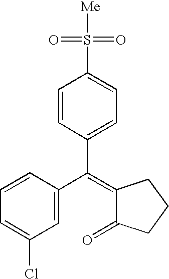 |
| | 3-[(3-chloro-phenyl)-(4-methanesulfonyl-phenyl)- |
| | methylene]-dihydro-furan-2-one or BMS-347070; |
| B-75 | 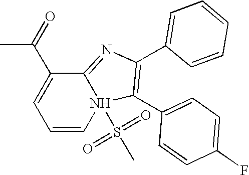 |
| | 8-acetyl-3-(4-fluorophenyl)-2-(4- |
| | methylsulfonyl)pbenyl-imidazo(1,2-a)pyridine; |
| B-76 | 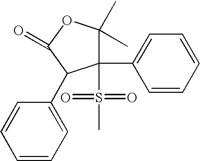 |
| | 5,5-dimethyl-4-(4-methylsulfonyl)phenyl-3- |
| | phenyl-2-(5H)-furanone; |
| B-77 | 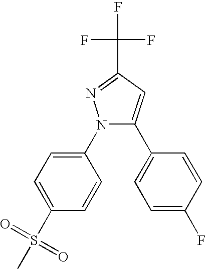 |
| | 5-(4-fluorophenyl)-1-[4-(methylsulfonyl) |
| | phenyl]-3-(trifluoromethyl)pyrazole; |
| B-78 | 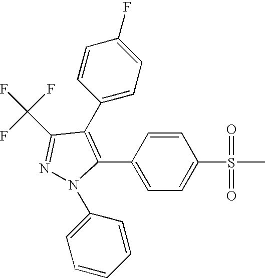 |
| | 4-(4-fluorophenyl)-5-[4-(methylsulfonyl) |
| | phenyl]-1-phenyl-3-(trifluoromethyl)pyrazole; |
| B-79 | 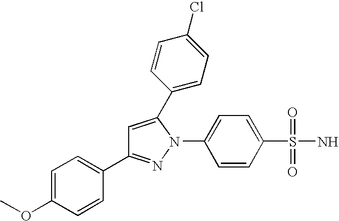 |
| | 4-(5-(4-chlorophenyl)-3-(4-methoxyphenyl)-1H- |
| | pyrazol-1-yl)benzenesulfonamide; |
| B-80 | 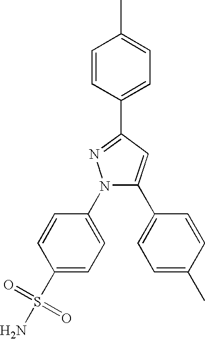 |
| | 4-(3,5-bis(4-methylphenyl)-1H-pyrazol-1- |
| | yl)benzenesulfonamide; |
| B-81 | 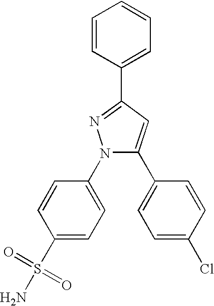 |
| | 4-(5-(4-chlorophenyl)-3-phenyl-1H-pyrazol-1- |
| | yl)benzenesulfonamide; |
| B-82 | 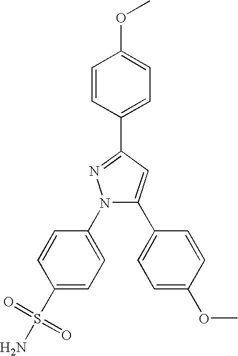 |
| | 4-(3,5-bis(4-methoxyphenyl)-1H-pyrazol-1-yl) |
| | benzenesulfonamide; |
| B-83 | 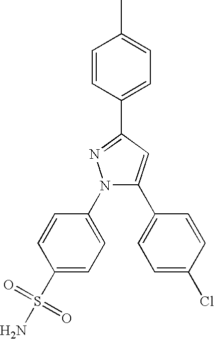 |
| | 4-(5-(4-chlorophenyl)-3-(4-methylphenyl)-1H- |
| | pyrazol-1-yl)benzenesulfonamide; |
| B-84 | 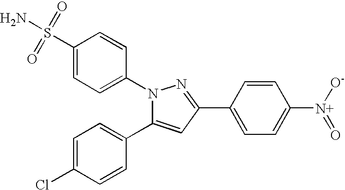 |
| | 4-(5-(4-chlorophenyl)-3-(4-nitrophenyl)-1H- |
| | pyrazol-1-yl)benzenesulfonamide; |
| B-85 | 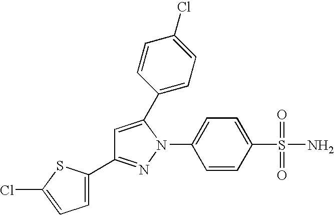 |
| | 4-(5-(4-chlorophenyl)-3-(5-chloro-2-thienyl)- |
| | 1H-pyrazol-1-yl)benzenesulfonamide; |
| B-86 | 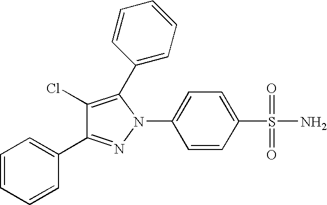 |
| | 4-(4-chloro-3,5-diphenyl-1H-pyrazol-1- |
| | yl)benzenesulfonamide; |
| B-87 | 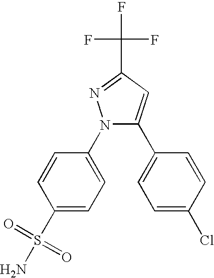 |
| | 4-[5-(4-chlorophenyl)-3-(trifluoromethyl)-1H- |
| | pyrazol-1-yl]benzenesulfonamide; |
| B-88 | 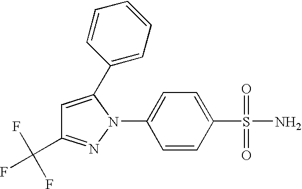 |
| | 4-[5-phenyl-3-(trifluoromethyl)-1H-pyrazol-1- |
| | yl]benzenesulfonamide; |
| B-89 | 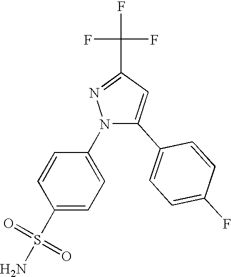 |
| | 4-[5-(4-fluorophenyl)-3-(trifluoromethyl)-1H- |
| | pyrazol-1-yl]benzenesulfonamide; |
| B-90 | 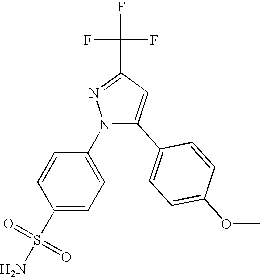 |
| | 4[5-(4-methoxyphenyl)-3-(trifluoromethyl)- |
| | 1H-pyrazol-1-yl]benzenesulfonamide; |
| B-91 | 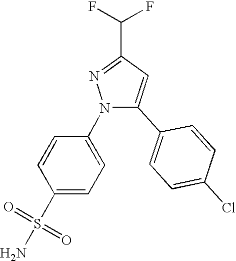 |
| | 4-[5-(4-chlorophenyl)-3-(difluoromethyl)- |
| | 1H-pyrazol-1-yl]benzenesulfonamide; |
| B-92 | 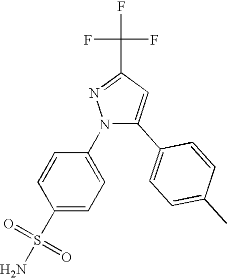 |
| | 4-[5-(4-methylphenyl)-3-(trifluoromethyl)- |
| | 1H-pyrazol-1-yl]benzenesulfonamide; |
| B-93 | 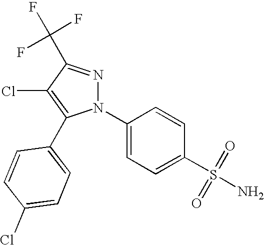 |
| | 4-[4-chloro-5-(4-chlorophenyl)-3-(trifluoromethyl)- |
| | 1H-pyrazol-1-yl]benzenesulfonamide; |
| B-94 | 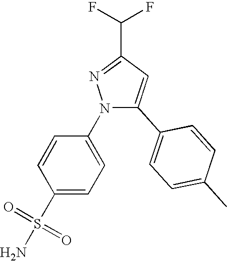 |
| | 4-[3-(difluoromethyl)-5-(4-methylphenyl)- |
| | 1H-pyrazol-1-yl]benzenesulfonamide; |
| B-95 | 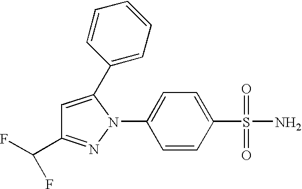 |
| | 4-[3-(difluoromethyl)-5-phenyl-1H- |
| | pyrazol-1-yl]benzenesulfonamide; |
| B-96 | 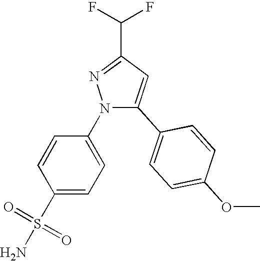 |
| | 4-[3-(difluoromethyl)-5-(4-methoxyphenyl)-1H- |
| | pyrazol-1-yl]benzenesulfonamide; |
| B-97 | 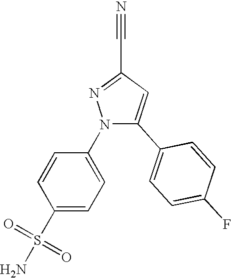 |
| | 4-[3-cyano-5-(4-fluorophenyl)-1H-pyrazol-1- |
| | yl]benzenesulfonamide; |
| B-98 | 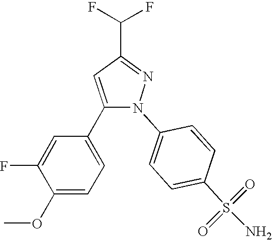 |
| | 4-[3-(difluoromethyl)-5-(3-fluoro-4- |
| | methoxyphenyl)-1H-pyrazol-1-yl] |
| | benzenesulfonamide; |
| B-99 | 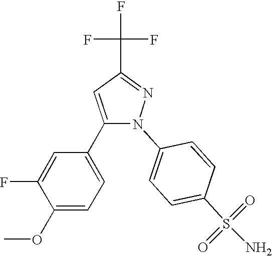 |
| | 4-[5-(3-fluoro-4-methoxyphenyl)-3- |
| | (trifluoromethyl)-1H-pyrazol-1-yl] |
| | benzenesulfonamide; |
| B-100 | 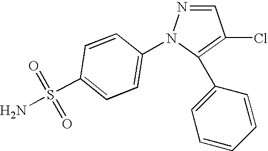 |
| | 4-[4-chloro-5-phenyl-1H-pyrazol-1- |
| | yl]benzenesulfonamide; |
| B-101 | 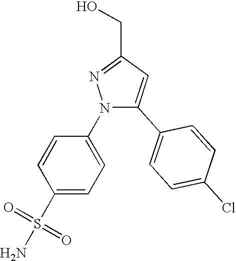 |
| | 4-[5-(4-chlorophenyl)-3-(hydroxymethyl)- |
| | 1H-pyrazol-1-yl]benzenesulfonamide; |
| B-102 | 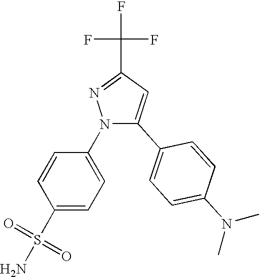 |
| | 4-[5-(4-(N,N-dimethylamino)phenyl)-3- |
| | (trifluoromethyl)-1H-pyrazol-1- |
| | yl]benzenesulfonamide; |
| B-103 | 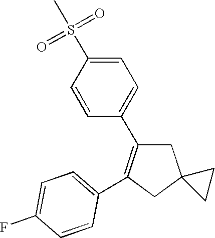 |
| | 5-(4-fluorophenyl)-6-[4- |
| | (methylsulfonyl)phenyl]spiro[2.4]hept-5-ene; |
| B-104 | 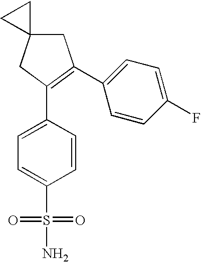 |
| | 4-[6-(4-fluorophenyl)spiro[2.4]hept-5- |
| | en-5-yl]benzenesulfonamide; |
| B-105 | 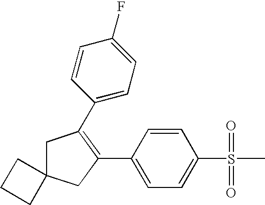 |
| | 6-(4-fluorophenyl)-7-[4-methylsulfonyl) |
| | phenyl]spiro[3.4]oct-6-ene; |
| B-106 | 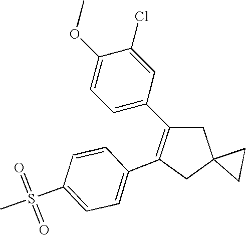 |
| | 5-(3-chloro-4-methoxypbenyl)-6-[4- |
| | (methylsulfonyl)phenyl]spira[2.4]hept-5-ene; |
| B-107 | 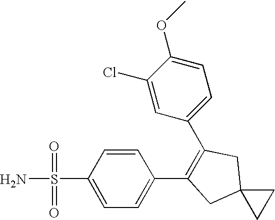 |
| | 4-[6-(3-chloro-4-methoxyphenyl)spiro[2.4] |
| | hept-5-en-5-yl]benzenesulfonamide; |
| B-108 | 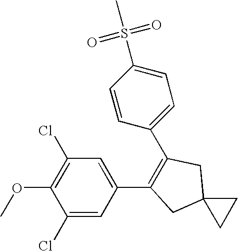 |
| | 5-(3,5-dichloro-4-methoxyphenyl)-6-[4- |
| | (methylsulfonyl)phenyl]spiro[2.4]hept- |
| | 5-ene; |
| B-109 | 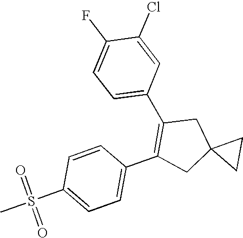 |
| | 5-(3-chloro-4-fluorophenyl)-6-[4- |
| | (methylsulfonyl)phenyl]spiro[2.4]hept- |
| | 5-ene; |
| B-110 | 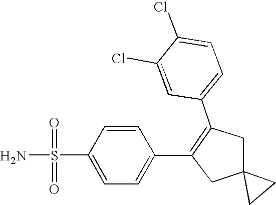 |
| | 4-[6-(3,4-dichlorophenyl)spiro[2.4] |
| | hept-5-en-5-yl]benzenesulfonamide; |
| B-111 | 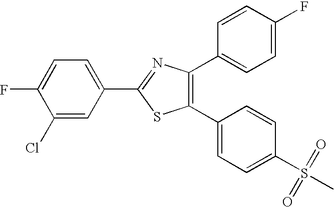 |
| | 2-(3-chloro-4-fluorophenyl)-4-(4- |
| | fluorophenyl)-5-(4- |
| | methylsulfonylphenyl)thiazole; |
| B-112 | 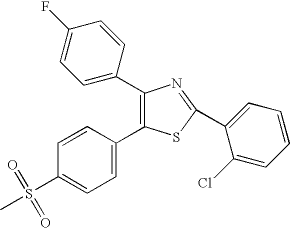 |
| | 2-(2-chlorophenyl)-4-(4-fluorophenyl)-5- |
| | (4-methylsulfonylphenyl)thiazole; |
| B-113 | 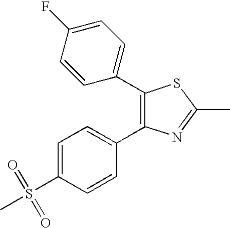 |
| | 5-(4-fluorophenyl)-4-(4- |
| | methylsulfonylphenyl)-2-methylthiazole; |
| B-114 | 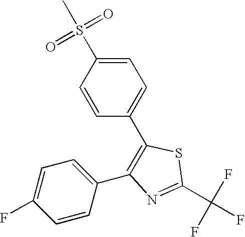 |
| | 4-(4-fluorophenyl)-5-(4- |
| | methylsulfonylphenyl)-2-trifluoromethylthiazole; |
| B-115 | 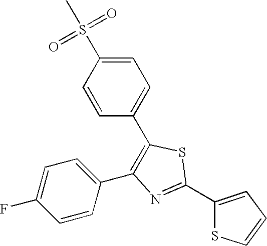 |
| | 4-(4-fluorophenyl)-5-(4-methylsulfonylphenyl)- |
| | 2-(2-thienyl)thiazole; |
| B-116 | 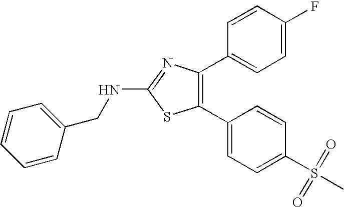 |
| | 4-(4-fluorophenyl)-5-(4-methylsulfonylphenyl)- |
| | 2-benzylaminothiazole; |
| B-117 | 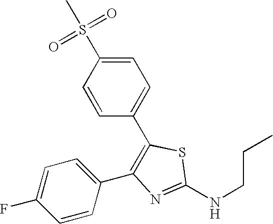 |
| | 4-(4-fluorophenyl)-5-(4-methylsulfonylphenyl)- |
| | 2-(1-propylamino)thiazole; |
| B-118 | 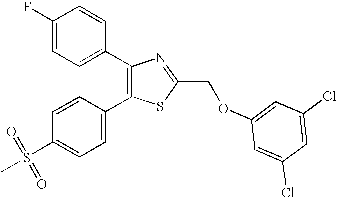 |
| | 2-((3,5-dichlorophenoxy)methyl)-4-(4- |
| | fluorophenyl)-5-[4-(methylsulfonyl) |
| | phenyl]thiazole; |
| B-119 | 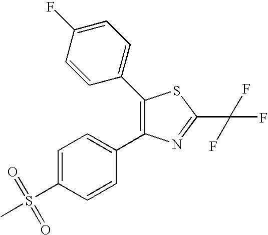 |
| | 5-(4-fluorophenyl)-4-(4- |
| | methylsulfonylphenyl)-2-trifluoromethylthiazole; |
| B-120 | 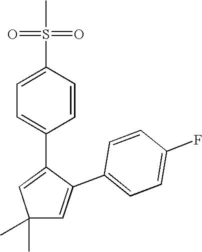 |
| | 1-methylsulfonyl-4-[1,1-dimethyl-4-(4- |
| | fluorophenyl)cyclopenta-2,4-dien-3-yl]benzene; |
| B-121 | 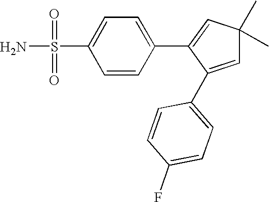 |
| | 4-[4-(4-fluorophenyl)-1,1- |
| | dimethylcyclopenta-2,4-dien-3-yl] |
| | benzenesulfonamide; |
| B-122 | 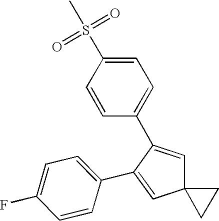 |
| | 5-(4-fluorophenyl)-6-[4- |
| | (methylsulfonyl)phenyl]spiro[2.4] |
| | hepta-4,6-diene; |
| B-123 | 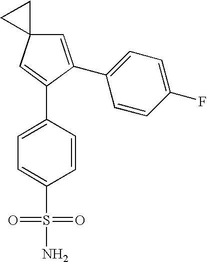 |
| | 4-[6-(4-fluorophenyl)spiro[2.4]hepta- |
| | 4,6-dien-5-yl]benzenesulfonamide; |
| B-124 | 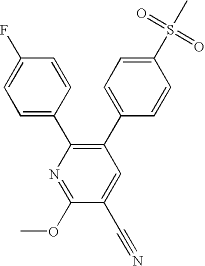 |
| | 6-(4-fluorophenyl)-2-methoxy-5-[4- |
| | (methylsulfonyl)phenyl]- |
| | pyridine-3-carbonitrile; |
| B-125 | 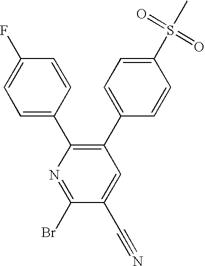 |
| | 2-bromo-6-(4-fluorophenyl)-5-[4- |
| | (methylsulfonyl)phenyl]- |
| | pyridine-3-carbonitrile; |
| B-126 | 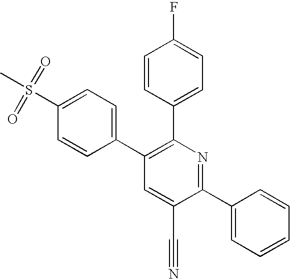 |
| | 6-(4-fluorophenyl)-5-[4- |
| | (methylsulfonyl)phenyl]-2-phenyl- |
| | pyridine-3-carbonitrile; |
| B-127 | 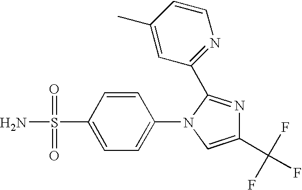 |
| | 4-[2-(4-methylpyridin-2-yl)-4- |
| | (trifluoromethyl)-1H-imidazol-1-yl] |
| | benzenesulfonamide; |
| B-128 | 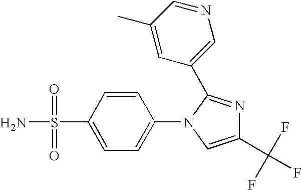 |
| | 4-[2-(5-methylpyridin-3-yl)-4- |
| | (trifluoromethyl)-1H-imidazol-1-yl] |
| | benzenesulfonamide; |
| B-129 | 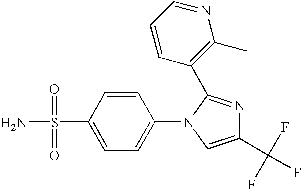 |
| | 4-[2-(2-methylpyridin-3-yl)-4- |
| | (trifluoromethyl)-1H-imidazol-1-yl] |
| | benzenesulfonamide; |
| B-130 | 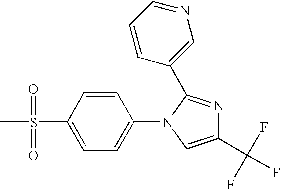 |
| | 3-[1-[4-(methylsulfonyl)phenyl]-4- |
| | (trifluoromethyl)-1H-imidazol-2-yl]pyridine; |
| B-131 | 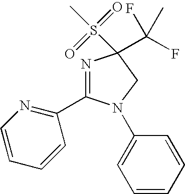 |
| | 2-[1-[4-(methylsulfonyl)phenyl-4- |
| | (trifluoromethyl)]-1H-imidazol-2-yl]pyridine; |
| B-132 | 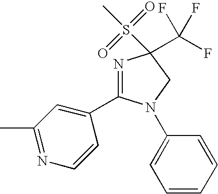 |
| | 2-methyl-4-[1-[4-(methylsulfonyl)phenyl-4- |
| | (trifluoromethyl)]-1H-imidazol-2- |
| | yl]pyridine; |
| B-133 | 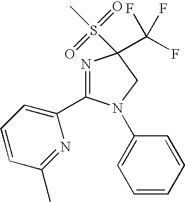 |
| | 2-methyl-6-[1-[4-(methylsulfonyl)phenyl-4- |
| | (trifluoromethyl)]-1H-imidazol-2-yl]pyridine; |
| B-134 | 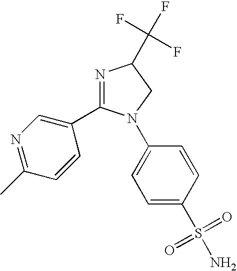 |
| | 4-[2-(6-methylpyridin-3-yl)-4-(trifluoromethyl)- |
| | 1H-imidazol-1-yl]benzenesulfonamide; |
| B-135 | 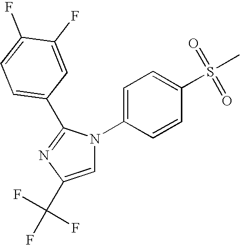 |
| | 2-(3,4-difluorophenyl)-1-[4-(methylsulfonyl) |
| | phenyl]-4-(trifluoromethyl)-1H-imidazole; |
| B-136 | 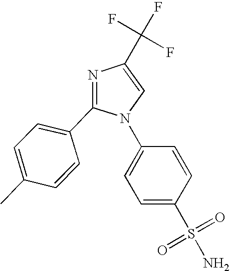 |
| | 4-[2-(4-methylphenyl)-4-(trifluoromethyl)- |
| | 1H-imidazol-1-yl]benzenesulfonamide; |
| B-137 | 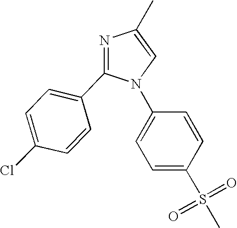 |
| | 2-(4-chlorophenyl)-1-[4-(methylsulfonyl) |
| | phenyl]-4-methyl-1H-imidazole; |
| B-138 | 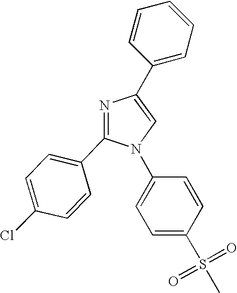 |
| | 2-(4-chlorophenyl)-1-[4-(methylsulfonyl) |
| | phenyl]-4-phenyl-1H-imidazole; |
| B-139 | 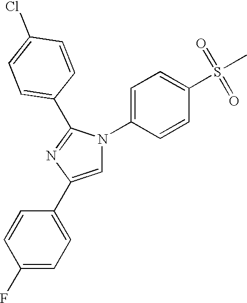 |
| | 2-(4-chlorophenyl)-4-(4-fluorophenyl)-1-[4- |
| | (methylsulfonyl)phenyl]-1H-imidazole; |
| B-140 | 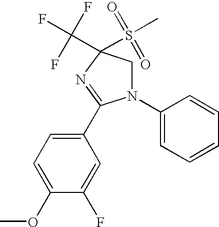 |
| | 2-(3-fluoro-4-methoxyphenyl)-1-[4- |
| | (methylsulfonyl)phenyl-4- |
| | (trifluoromethyl)]-1H-imidazole; |
| B-141 | 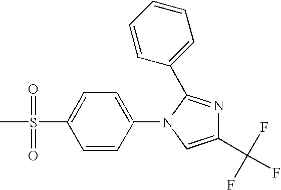 |
| | 1-[4-(methylsulfonyl)phenyl]-2-phenyl-4- |
| | trifluoromethyl-1H-imidazole; |
| B-142 | 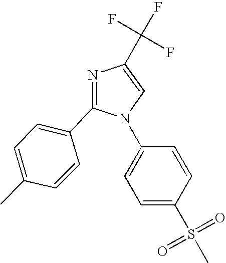 |
| | 2-(4-methylphenyl)-1-[4-(methylsulfonyl) |
| | phenyl]-4-trifluoromethyl-1H-imidazole; |
| B-143 | 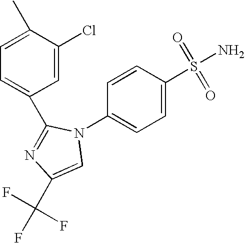 |
| | 4-[2-(3-chloro-4-methylphenyl)-4- |
| | (trifluoromethyl)-1H-imidazol-1- |
| | yl]benzenesulfonamide; |
| B-144 | 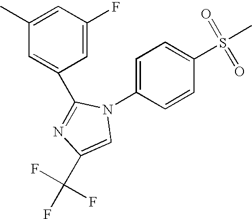 |
| | 2-(3-fluoro-5-methylphenyl)-1-[4- |
| | (methylsulfonyl)phenyl]- |
| | 4-(trifluoromethyl)-1H-imidazole; |
| B-145 | 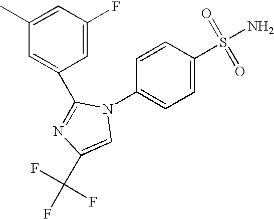 |
| | 4-[2-(3-fluoro-5-methylphenyl)-4- |
| | (trifluoromethyl-1H-imidazole-1- |
| | yl]benzenesulfonamide; |
| B-146 | 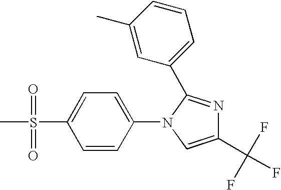 |
| | 2-(3-methylphenyl)-1-[4-(methylsulfonyl) |
| | phenyl]-4-trifluoromethyl-1H-imidazole; |
| B-147 | 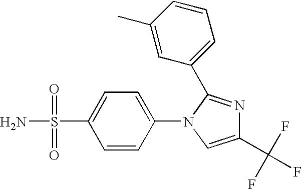 |
| | 4-[2-(3-methylphenyl)-4-trifluoromethyl- |
| | 1H-imidazol-1-yl]benzenesulfonamide; |
| B-148 | 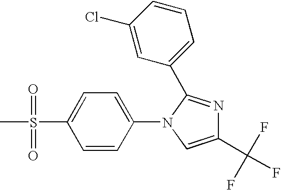 |
| | 1-[4-(methylsulfonyl)phenyl]-2-(3- |
| | chlorophenyl)-4-trifluoromethyl-1H-imidazole |
| B-149 | 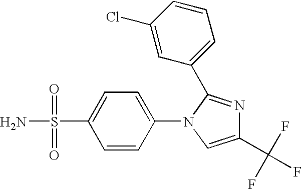 |
| | 4-[2-(3-chlorophenyl)-4-trifluoromethyl-1H- |
| | imidazol-1-yl]benzenesulfonamide; |
| B-150 | 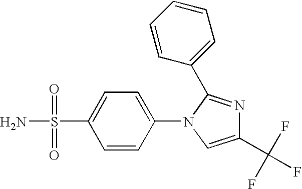 |
| | 4-[2-phenyl-4-trifluoromethyl-1H-imidazol-1- |
| | yl]benzenesulfonamide; |
| B-151 | 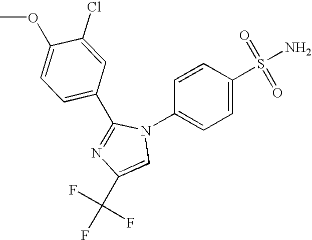 |
| | 4-[2-(4-methoxy-3-chlorophenyl)-4- |
| | trifluoromethyl-1H-imidazol-1- |
| | yl]benzenesulfonamide; |
| B-152 | 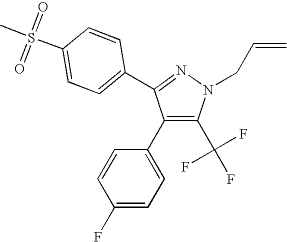 |
| | 1-allyl-4-(4-fluorophenyl)-3-[4- |
| | (methylsulfonyl)phenyl]-5- |
| | (trifluoromethyl)-1H-pyrazole; |
| B-153 | 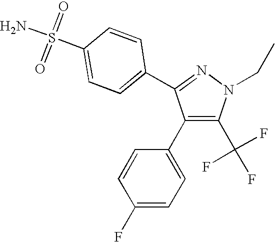 |
| | 4-[1-ethyl-4-(4-fluorophenyl)-5- |
| | (trifluoromethyl)-1H-pyrazol-3-yl] |
| | benzenesulfonamide; |
| B-154 | 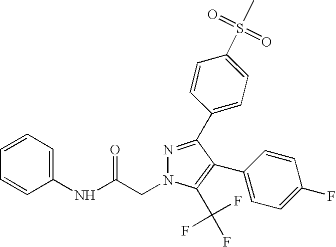 |
| | N-phenyl-[4-(4-fluorophenyl)-3-[4- |
| | (methylsulfonyl)phenyl]- |
| | 5-(trifluoromethyl)-1H-pyrazol-1-yl]acetamide; |
| B-155 | 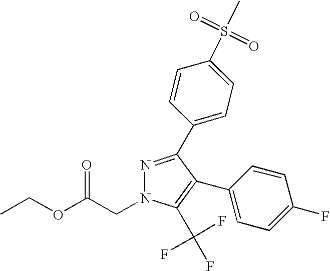 |
| | ethyl[4-(4-fluorophenyl)-3-[4- |
| | (methylsulfonyl)phenyl]-5-(trifluoromethyl)- |
| | 1H-pyrazol-1-yl]acetate; |
| B-156 | 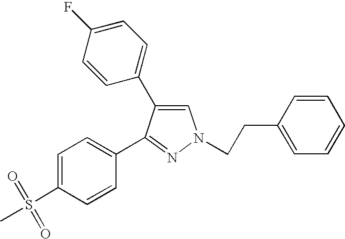 |
| | 4-(4-fluorophenyl)-3-[4-(methylsulfonyl) |
| | phenyl]-1-(2-phenylethyl)-1H-pyrazole; |
| B-157 | 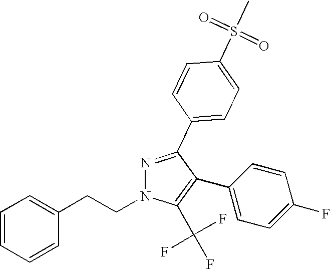 |
| | 4-(4-fluorophenyl)-3-[4-(methylsulfonyl) |
| | phenyl]-1-(2-phenylethyl )-5- |
| | (trifluoromethyl)pyrazole; |
| B-158 | 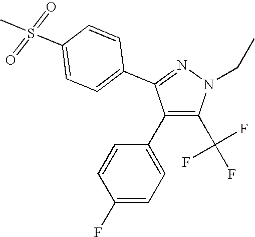 |
| | 1-ethyl-4-(4-fluorophenyl)-3-[4- |
| | methylsulfonyl)phenyl]-5-(trifluoromethyl)- |
| | 1H-pyrazole; |
| B-159 | 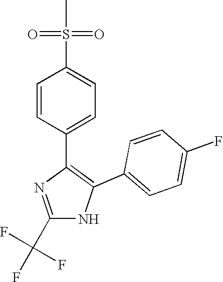 |
| | 5-(4-fluorophenyl)-4-(4-methylsulfonylphenyl)- |
| | 2-trifluoromethyl-1H-imidazole; |
| B-160 | 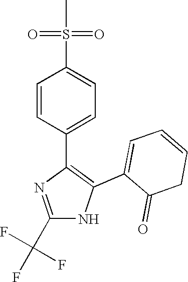 |
| | 4-[4-(methylsulfonyl)phenyl]-5-(2- |
| | thiophenyl)-2-(trifluoromethyl)-1H-imidazole; |
| B-161 | 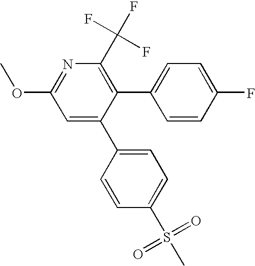 |
| | 5-(4-fluorophenyl)-2-methoxy-4-[4- |
| | (methylsulfonyl)phenyl]-6-(trifluoromethyl)pyridine; |
| B-162 | 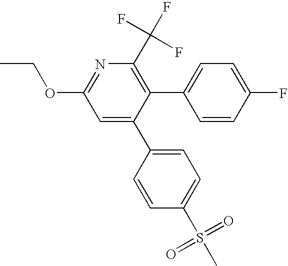 |
| | 2-ethoxy-5-(4-fluorophenyl)-4-[4- |
| | (methylsulfonyl)phenyl]-6- |
| | (trifluoromethyl)pyridine; |
| B-163 | 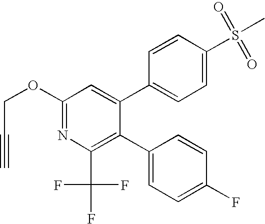 |
| | 5-(4-fluorophenyl)-4-[4-(methylsulfonyl) |
| | phenyl]-2-(2-propynyloxy)-6- |
| | (trifluoromethyl)pyridine; |
| B-164 | 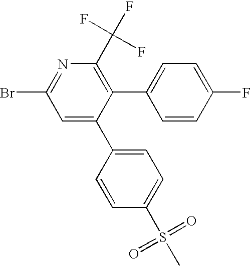 |
| | 2-bromo-5-(4-fluorophenyl)-4-[4- |
| | (methylsulfonyl)phenyl]-6- |
| | (trifluoromethyl)pyridine; |
| B-165 | 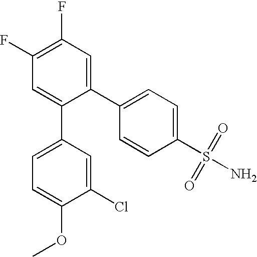 |
| | 4-[2-(3-chloro-4-methoxyphenyl)-4,5- |
| | difluorophenyl]benzenesulfonamide; |
| B-166 | 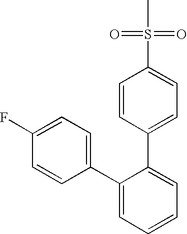 |
| | 1-(4-fluorophenyl)-2-[4-methylsulfonyl) |
| | phenyl]benzene; |
| B-167 | 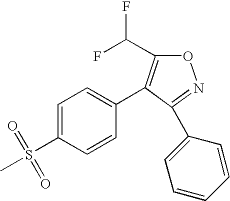 |
| | 5-difluoromethyl-4-(4-methylsulfonylphenyl)- |
| | 3-phenylisoxazole; |
| B-168 | 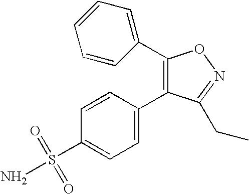 |
| | 4-[3-ethyl-5-phenylisoxazol-4-yl] |
| | benzenesulfonamide; |
| B-169 | 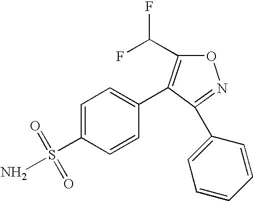 |
| | 4-[5-difluoromethyl-3-phenylisoxazol- |
| | 4-yl]benzenesulfonamide; |
| B-170 | 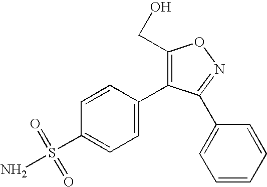 |
| | 4-[5-hydroxymethyl-3-phenylisoxazol- |
| | 4-yl]benzenesulfonamide; |
| B-171 | 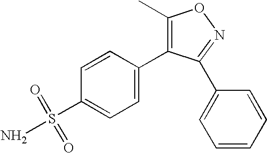 |
| | 4-[5-methyl-3-phenyl-isoxazol-4- |
| | yl]benzenesulfonamide; |
| B-172 | 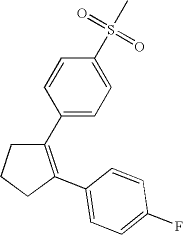 |
| | 1-[2-(4-fluorophenyl)cyclopenten-1- |
| | yl]-4-(methylsulfonyl)benzene; |
| B-173 | 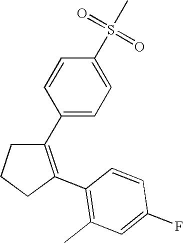 |
| | 1-[2-(4-fluoro-2-methylphenyl)cyclopenten- |
| | 1-yl]-4-(methylsulfonyl)benzene; |
| B-174 | 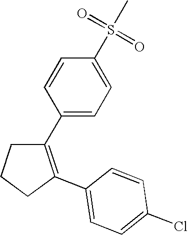 |
| | 1-[2-(4-chlorophenyl)cyclopenten-1- |
| | yl]-4-(methylsulfonyl)benzene; |
| B-175 | 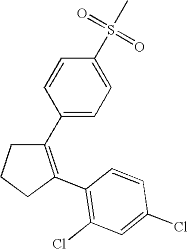 |
| | 1-[2-(2,4-dichlorophenyl)cyclopenten-1- |
| | yl]-4-(methylsulfonyl)benzene; |
| B-176 | 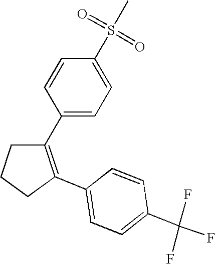 |
| | 1-[2-(4-trifloromethylphenyl)cyclopenten- |
| | 1-yl)-4-(methylsulfonyl)benzene; |
| B-177 | 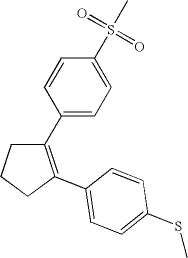 |
| | 1-[2-(4-methylthiophenyl)cyclopenten-1- |
| | yl]-4-(methylsulfonyl)benzene; |
| B-178 | 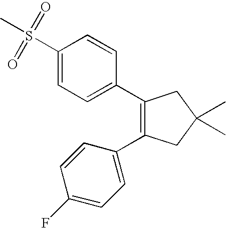 |
| | 1-[2-(4-fluorophenyl)-4,4-dimethylcyclopenten- |
| | 1-yl]-4-(methylsulfonyl)benzene; |
| B-179 | 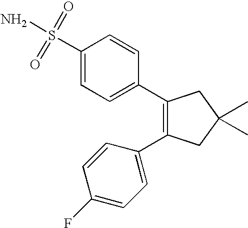 |
| | 4-[2-(4-fluorophenyl)-4,4- |
| | dimethylcyclopenten-1-yl]benzenesulfonamide; |
| B-180 | 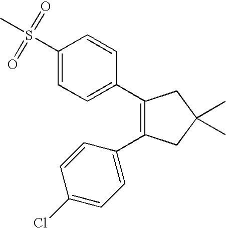 |
| | 1-[2-(3-chlorophenyl)-4,4- |
| | dimethylcyclopenten-1-yl]-4- |
| | (methylsulfonyl)benzene; |
| B-181 | 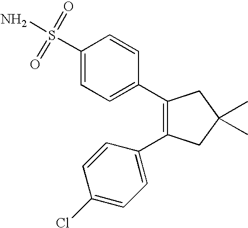 |
| | 4-[2-(4-chlorophenyl)-4,4- |
| | dimethylcyclopenten-1-yl]benzenesulfonamide; |
| B-182 | 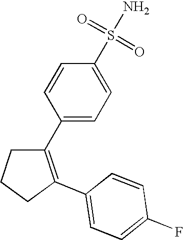 |
| | 4-[2-(4-fluorophenyl)cyclopenten-1- |
| | yl]benzenesulfonamide; |
| B-183 | 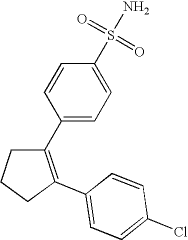 |
| | 4-[2-(4-chlorophenyl)cyclopenten-1- |
| | yl]benzenesulfonamide; |
| B-184 | 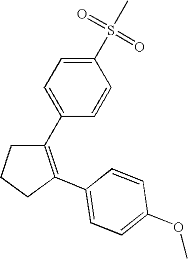 |
| | 1-[2-(4-methoxyphenyl)cyclopenten-1- |
| | yl]-4-(methylsulfonyl)benzene; |
| B-185 | 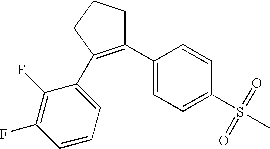 |
| | 1-[2-(2,3-difluorophenyl)cyclopenten- |
| | 1-yl]-4-(methylsulfonyl)benzene; |
| B-186 | 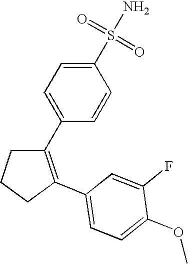 |
| | 4-[2-(3-fluoro-4-methoxyphenyl)cyclopenten- |
| | 1-yl]benzenesulfonamide; |
| B-187 | 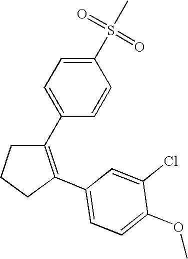 |
| | 1-[2-(3-chloro-4-methoxyphenyl)cyclopenten- |
| | 1-yl]-4-(methylsulfonyl)benzene; |
| B-188 | 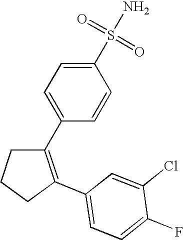 |
| | 4-[2-(3-chloro-4-fluorophenyl)cyclopenten- |
| | 1-yl]benzenesulfonamide; |
| B-189 | 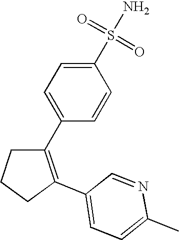 |
| | 4-[2-(2-methylpyridin-5-yl)cyclopenten-1- |
| | yl]benzenesulfonamide; |
| B-190 | 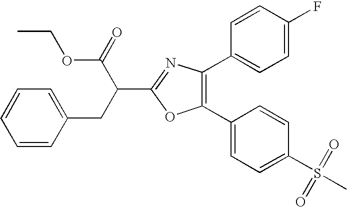 |
| | ethyl 2-[4-(4-fluorophenyl)-5-[4- |
| | (methylsulfonyl)phenyl]oxazol-2- |
| | yl]-2-benzyl-acetate; |
| B-191 | 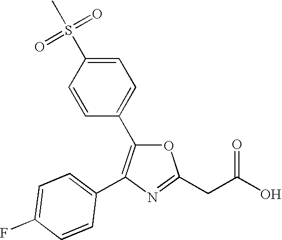 |
| | 2-[4-(4-fluorophenyl)-5-[4- |
| | (methylsulfonyl)phenyl]oxazol-2- |
| | yl]acetic acid; |
| B-192 | 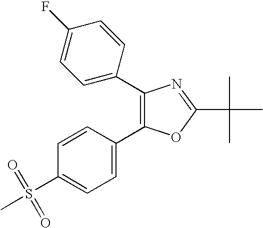 |
| | 2-(tert-butyl)-4-(4-fluorophenyl)-5- |
| | [4-(methylsulfonyl)phenyl]oxazole; |
| B-193 | 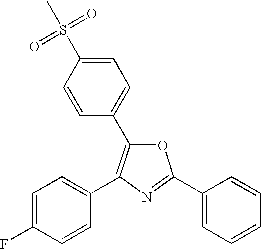 |
| | 4-(4-fluorophenyl)-5-[4-(methylsulfonyl) |
| | phenyl]-2-phenyloxazole; |
| B-194 | 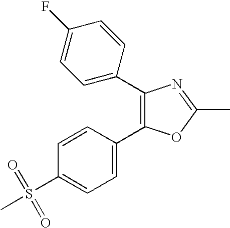 |
| | 4-(4-fluorophenyl)-2-methyl-5-[4- |
| | (methylsulfonyl)phenyl]oxazole; |
| B-195 | 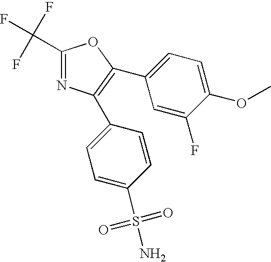 |
| | 4-[5-(3-fluoro-4-methoxyphenyl)-2- |
| | trifluoromethyl-4-oxazolyl]benzenesulfonamide; |
| B-196 | 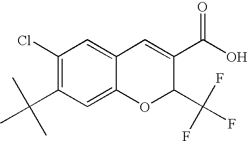 |
| | 6-chloro-7-(1,1-dimethylethyl)-2- |
| | trifluoromethyl-2H-1-benzopyran-3- |
| | carboxylic acid; |
| B-197 | 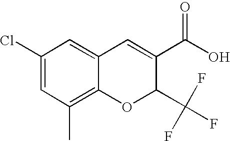 |
| | 6-chloro-8-methyl-2-trifluoromethyl-2H- |
| | 1-benzopyran-3-carboxylic acid; |
| B-198 | 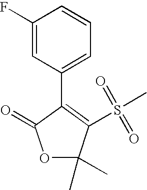 |
| | 5,5-dimethyl-3-(3-fluorophenyl)-4- |
| | methylsulfonyl-2(5H)-furanone; |
| B-199 | 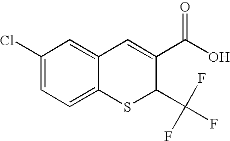 |
| | 6-chloro-2-trifluoromethyl-2H-1- |
| | benzothiopyran-3-carboxylic acid; |
| B-200 | 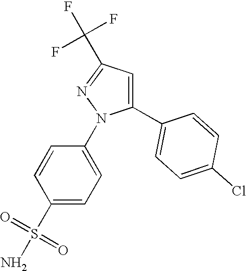 |
| | 4-[5-(4-chlorophenyl)-3-(trifluoromethyl)- |
| | 1H-pyrazol-1-yl]benzenesulfonamide; |
| B-201 | 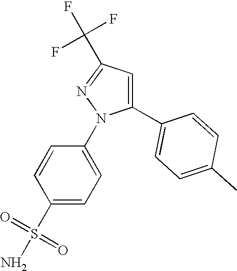 |
| | 4-[5-(4-methylphenyl)-3-(trifluoromethyl)- |
| | 1H-pyrazol-1-yl]benzenesulfonamide; |
| B-202 | 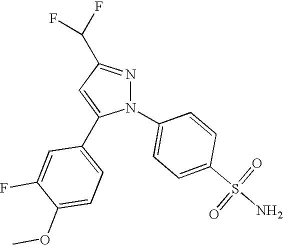 |
| | 4-[5-(3-fluoro-4-methoxyphenyl)-3- |
| | (difluoromethyl)-1H-pyrazol-1- |
| | yl]benzenesulfonamide; |
| B-203 | 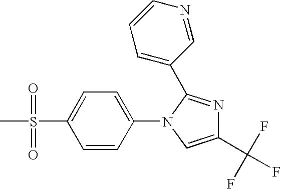 |
| | 3-[1-[4-(methylsulfonyl)phenyl]-4- |
| | trifluoromethyl-1H-imidazol-2-yl]pyridine; |
| B-204 | 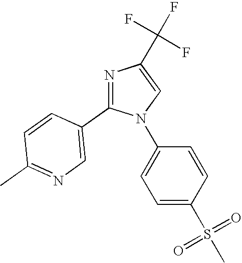 |
| | 2-methyl-5-[1-[4-(methylsulfonyl)phenyl]-4- |
| | trifluoromethyl-1H-imidazol-2-yl]pyridine; |
| B-205 | 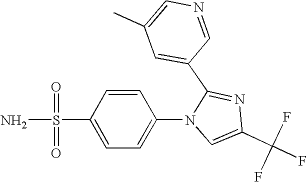 |
| | 4-[2-(5-methylpyridin-3-yl)-4-(trifluoromethyl)- |
| | 1H-imidazol-1-yl]benzenesulfonamide; |
| B-206 | 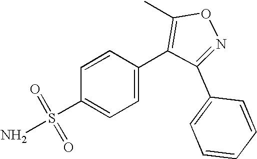 |
| | 4-[5-methyl-3-phenylisoxazol-4-yl] |
| | benzenesulfonamide; |
| B-207 | 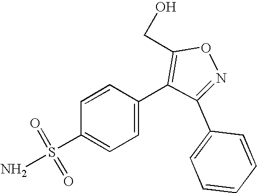 |
| | 4-[5-hydroxymethyl-3-phenylisoxazol-4- |
| | yl]benzenesulfonamide; |
| B-208 | 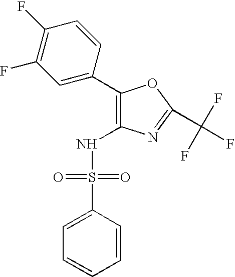 |
| | [2-trifluoromethyl-5-(3,4-difluorophenyl)- |
| | 4-oxazolyl]benzenesulfonamide; |
| B-209 | 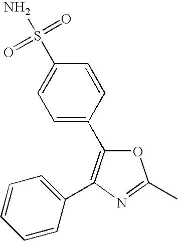 |
| | 4-[2-methyl-4-phenyl-5-oxazolyl] |
| | benzenesulfonamide; |
| B-210 | 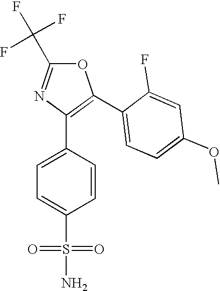 |
| | 4-[5-(2-fluoro-4-methoxyphenyl)-2- |
| | trifluoromethyl-4-oxazolyl] |
| | benzenesulfonamide; |
| B-211 | 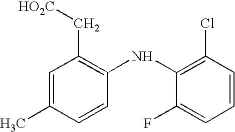 |
| B-212 | 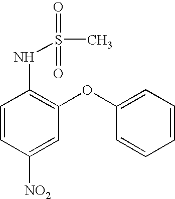 |
| | N-(4-nitro-2-phenoxy-phenyl)- |
| | methanesulfonamide or Nimesulide |
| B-213 | 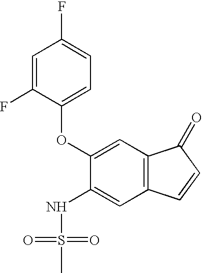 |
| | N-[6-(2,4-difluoro-phenoxy)-1-oxo-inden-5- |
| | yl]-methanesulfonamide or Flosulide |
| B-214 | 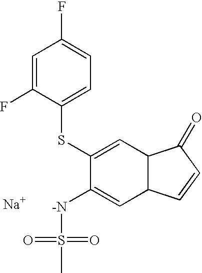 |
| | N-[6-(2,4-difluoro-phenylsulfanyl)-1-oxo- |
| | 1H-inden-5-yl]-methanesulfonamide, |
| | soldium salt, or L-745337 |
| B-215 | 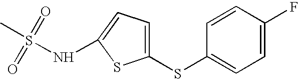 |
| | N-[5-(4-fluoro-phenylsulfanyl)-thiophen-2- |
| | yl]-methanesulfonamide or RWJ-63556 |
| B-216 | 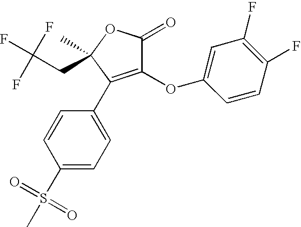 |
| | 3-(3,4-difluoro-phenoxy)-4-(4-methanesulfonyl- |
| | phenyl)-5-methyl-5-(2,2,2-trifluoro-ethyl)-5H- |
| | furan-2-one or L-784512 |
| B-217 | 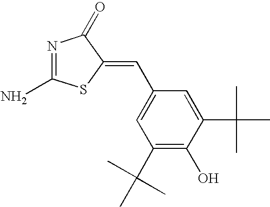 |
| | (5Z)-2-amino-5-[[3,5-bis(1,1-dimethylethyl)-4- |
| | hydroxyphenyl]methylene]-4(5H)- |
| | thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 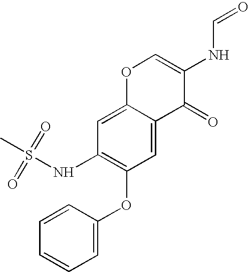 |
| | N-[3-(formylamino)-4-oxo-6-phenoxy-4H-1- |
| | benzopyran-7-yl]-methanesulfonanxide or T614 |
| B-225 | D-1367 |
| B-226 | L-748731 |
| B-227 | 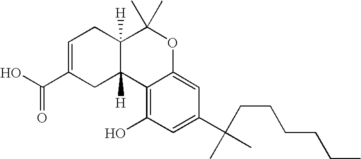 |
| | (6aR,10aR)-3-(1,1-dimethylheptyl)-6a,7,10,10a- |
| | tetrahydro-1-hydroxy-6,6-dimethyl-6H- |
| | dibenzo[b,d]pyran-9- |
| | carboxylic acid or CT3 |
| B-228 | CGP-28238 |
| B-229 | 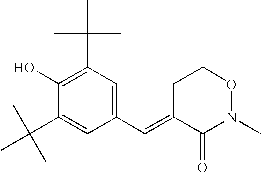 |
| | 4-[[3,5-bis(1,1-dimethylethyl)-4- |
| | hydroxyphenyl]methylene] |
| | dihydro-2-methyl-2H-1,2-oxazin-3(4H)- |
| | one or BF-389 |
| B-230 | GR-253035 |
| B-231 | 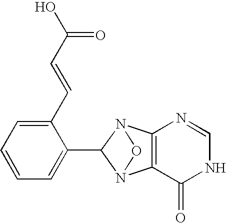 |
| | 2-(6-dioxo-9H-purin-8-yl)cinnamic acid |
| B-232 | S-2474 |
| B-233 | 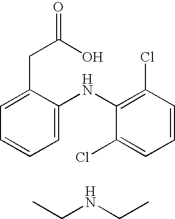 |
| B-234 | 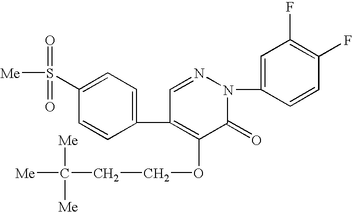 |
| B-235 | 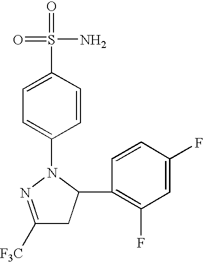 |
| B-236 | 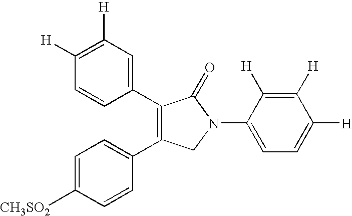 |
| B-237 | 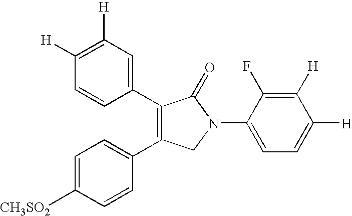 |
| B-238 | 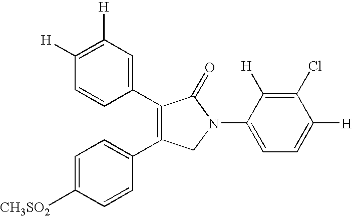 |
| B-239 | 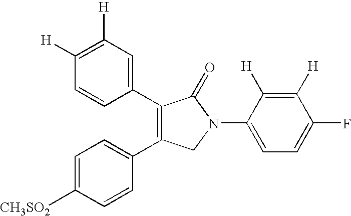 |
| B-240 | 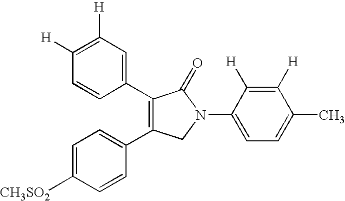 |
| B-241 | 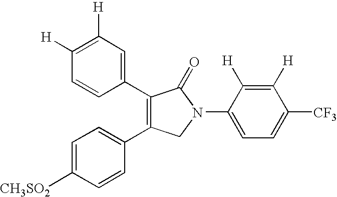 |
| B-242 | 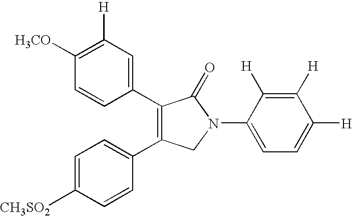 |
| B-243 | 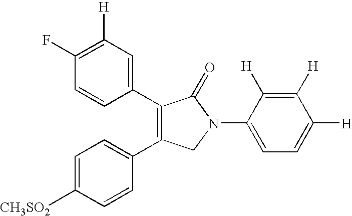 |
| B-244 | 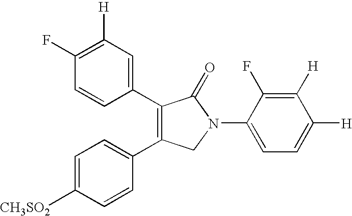 |
| B-245 | 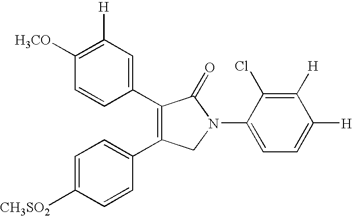 |
| B-246 | 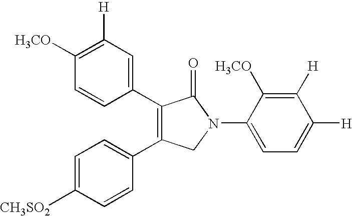 |
| B-247 | 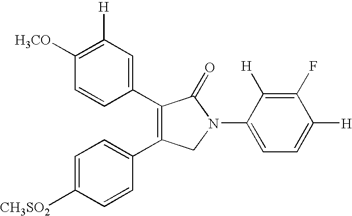 |
| B-248 | 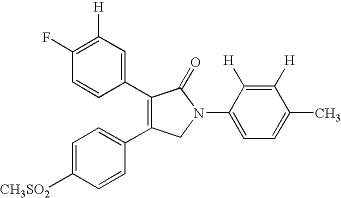 |
| B-249 | 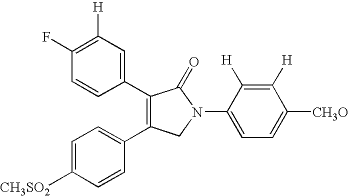 |
| B-250 | 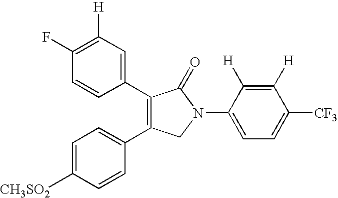 |
| B-251 | 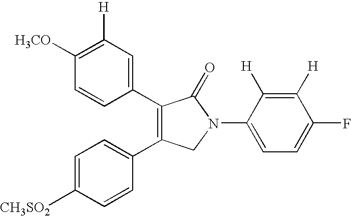 |
| B-252 | 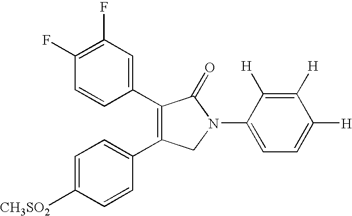 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day kg, and even more typically, from about 0.18 to about 0.4 mg/day·kg.

In still another embodiment, when the cyclooxygenase-2 selective inhibitor comprises etoricoxib, it is typical that the amount used is within a range of from about 0.5 to about 5 mg/day·kg, and even more typically, from about 0.8 to about 4 mg/day·kg.

Further, when the cyclooxygenase-2 selective inhibitor comprises celecoxib, it is typical that the amount used is within a range of from about 1 to about 20 mg/day·kg, even more typically, from about 1.4 to about 8.6 mg/day·kg, and yet more typically, from about 2 to about 3 mg/day·kg.

When the cyclooxygenase-2 selective inhibitor comprises valdecoxib, it is typical that the amount used is within a range of from about 0.1 to about 5 mg/day·kg, and even more typically, from about 0.8 to about 4 mg/day·kg.

In a further embodiment, when the cyclooxygenase-2 selective inhibitor comprises parecoxib, it is typical that the amount used is within a range of from about 0.1 to about 5 mg/day·kg, and even more typically, from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Sodium Ion Channel Blockers

In addition to a cyclooxygenase-2 selective inhibitor, the composition of the invention also comprises a therapeutically effective amount of a sodium ion channel blocker or a pharmaceutically acceptable salt or prodrug thereof. A number of sodium ion channel blockers may be employed in the present invention.

In one aspect of the invention, the sodium ion channel blocker is a member of the type IA antiarrythmic class of compounds. In one embodiment, the type IA antiarrythmic compound is selected from the group consisting of disopyramide, procainimide, and quinidine, or a pharmaceutically acceptable salt or prodrug thereof.

In another aspect of the invention, the sodium ion channel blocker is a member of the type IB antiarrythmic class of compounds. In one embodiment, the type IB antiarrythmic compound is selected from the group consisting of tocainide, mexiletene, lidocane, phenytoin, and fosphenytoin, or a pharmaceutically acceptable salt or prodrug thereof.

In still another aspect of the invention, the sodium ion channel blocker is a member of the type IC antiarrythmic class of compounds. In one embodiment, the type IC antiarrythmic compound is selected from the group consisting of flecainide, propafenone, and morcizine, or a pharmaceutically acceptable salt or prodrug thereof.

In a further embodiment, compounds that are useful for the sodium ion channel blocker or a pharmaceutically acceptable salt or prodrug thereof in connection with the present invention, the structures for which are set forth in Table 3C below, include, but are not limited to:

5,5-diphenyl-3-[(phosphonooxy)methyl]-2,4-imidazolidinedione disodium salt (C-1);

3,5-diamino-6-chloro-N-(diaminomethylene) pyrazinecarboxamide monohydrochloride, dihydrate (C-2);

p-amino-N-[2-(diethylamino)ethyl]-benzamide monohydrochloride (C-3);

Cinchonan-9-ol, 6′-methoxy-, (9S)-, sulfate (2:1) dihydrate (C-4);

2-(diethylamino)-N-(2,6-dimethylphenyl)-, monohydrochloride (C-5);

5H-Dibenz[b,f]azepine-5-carboxamide (C-6);

1-Methyl-2-(2,6-xyloxy)ethylamine hydrochloride (C-7);

N-(2-piperidinylmethyl)-2,5-bis(2,2,2-trifluoroethoxy)-monoacetate (C-8);

2-amino-6-(trifluoromethoxy) benzothiazole (C-9);

Octahydro-12-(hydroxymethyl)-2-imino-5,9,7,10 a-dimethano-10 aH-[1,3]dioxocino[6,5-d]pyrimidine-4,7,10,11,12-pentol (C-10);

Vinpocetine (C-11);

(E)-1-[bis(4-fluorophenyl)methyl]-4-(3-phenyl-2-propenyl)piperazine (C-12);

6-(2,3-dichlorophenyl)-1,2,4-triazine-3,5-diamine (C-13);

Anthopleurin-C (APE2-1) (C-14);

Pompilidotoxin (PMTX) (C-15);

ATX-II (C-16);

Flunarizine dihydrochloride (C-17);

I-Conotoxin GIIIB (C-18);

QX-222 (C-19);

QX-314 (C-20);

QX-222 Chloride Salt (C-21);

QX-314 Bromide Salt (C-22);

QX-314 Chloride Salt (C-23);

Tetrodotoxin (C-24);

Tetrodotoxin Citrate (C-25);

Spheroidine (C-26);

Maculotoxin (C-27);

TABLE 3C
EXAMPLES OF SODIUM ION CHANNEL BLOCKERS AS
EMBODIMENTS

| Compound | |
|---|---|
| Number | Structural Formula/Chemical Name |
| C-1 | 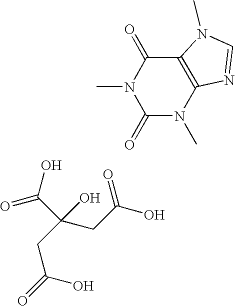 |
| | 5,5-diphenyl-3-[(phosphonooxy)methyl]-2,4-imi- |
| | dazolidinedione disodium salt |
| C-2 | 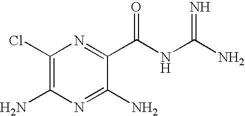 |
| | 3,5-diamino-6-chloro-N-(diaminomethylene)py- |
| | razinecarboxamide monohydrochloride, dihydrate |
| C-3 | 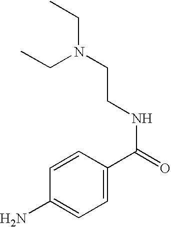 |
| | p-amino-N-[2-(diethylamino)ethyl]-benzamide |
| | monohydrochloride |
| C-4 | 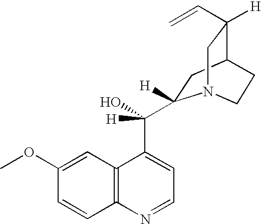 |
| | Cinchonan-9-ol, 6′-methoxy-, (9S)-, sulfate(2:1) |
| | dihydrate |
| C-5 | 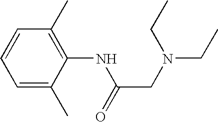 |
| | 2-(diethylamino)-N-(2,6-dimethylphenyl)-, |
| | monohydrochloride |
| C-6 | 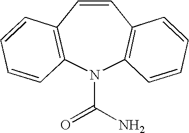 |
| | 5H-Dibenz[b,f]azepine-5-carboxamide |
| C-7 | 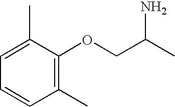 |
| | 1-Methyl-2-(2,6-xyloxy)ethylamine hydrochloride |
| C-8 | 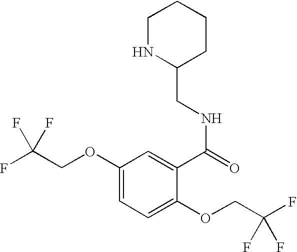 |
| | N-(2-piperidinylmethyl)-2,5-bis(2,2,2-trifluoro- |
| | ethoxy)-monoacetate |
| C-9 | 2-amino-6-(trifluoromethoxy)benzothiazole |
| C-10 | 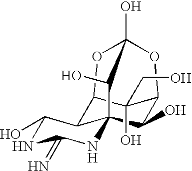 |
| | Octahydro-12-(hydroxymethyl)-2-imino-5,9,7,10a-di- |
| | methano-10aH-[1,3]dioxocino[6,5-d]pyrimi- |
| | dine-4,7,10,11,12-pentol |
| C-11 | 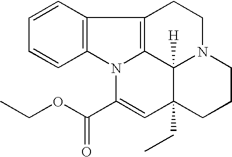 |
| | Vinpocetine |
| C-12 | 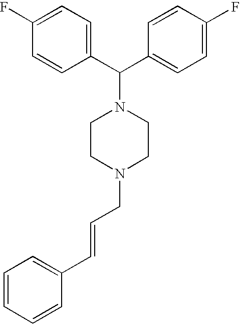 |
| | (E)-1-[bis(4-fluorophenyl)methyl]-4-(3-phenyl-2-pro- |
| | penyl)piperazine |
| C-13 | 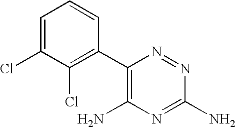 |
| | 6-(2,3-dichlorophenyl)-1,2,4-triazine-3,5-diamine |
| C-14 | Anthopleurin-C (APE2-1) |
| C-15 | Pompilidotoxin (PMTX) |
| C-16 | ATX-II |
| C-17 | Flunarizine dihydrochloride |
| C-18 | I-Conotoxin GIIIB |
| C-19 | QX-222 |
| C-20 | QX-314 |
| C-21 | QX-222 Chloride Salt |
| C-22 | QX-314 Bromide Salt |
| C-23 | QX-314 Chloride Salt |
| C-24 | Tetrodotoxin |
| C-25 | Tetrodotoxin Citrate |
| C-26 | Spheroidine |
| C-27 | Maculotoxin |

Generally speaking, the pharmacokinetics of the particular agent to be administered will dictate the most preferred method of administration and dosing regiment. The sodium ion channel blocker can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the sodium ion channel blocker can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

In another embodiment, the sodium ion channel blocker can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amounts of compound or drug can and will vary according to the specific composition being utilized, the mode of administration and the age, weight and condition of the subject. Dosages for a particular individual subject can be determined by one of ordinary skill in the art using conventional considerations. But in general, the amount of sodium ion channel blocker will be between about 0.5 to about 1000 milligrams per day and more typically, between about 2.5 to about 750 milligrams per day and even more typically, between about 5.0 to about 500 milligrams per day. The daily dose can be administered in one to four doses per day.

By way of example, in one embodiment when the sodium ion channel blocker is amiloride administered in a controlled release dosage form, the amount administered daily is typically from about 5 to about 20 milligrams per day administered in two to four doses per day. In an alternative of this embodiment, when the sodium ion channel blocker is lidocaine administered in a controlled release dosage form, the amount administered is also from about 5 to about 500 milligrams per day, administered in two to four doses per day.

By way of further example, in another embodiment when the sodium ion channel blocker is quinidine the amount administered daily is typically from about 200 to about 600 milligrams per day, administered in two to four doses per day. In an alternative of this embodiment, when the sodium ion channel blocker is mexiletine the amount administered daily is typically from about 500 to about 900 milligrams per day, administered in two to four doses per day.

By way of still further example, in another embodiment when the sodium ion channel blocker is flecainide the amount administered daily is typically from about 100 to about 300 milligrams per day, administered in two to four doses per day. In an alternative of this embodiment, when the sodium ion channel blocker is lamotrigine the amount administered daily is typically from about 0.6 to about 500 milligrams per day, administered in two to four doses per day.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The timing of the administration of the cyclooxygenase-2 selective inhibitor in relation to the administration of the sodium ion channel blocker may also vary from subject to subject. In one embodiment, the cyclooxygenase-2 selective inhibitor and sodium ion channel blocker may be administered substantially simultaneously, meaning that both agents may be administered to the subject at approximately the same time. For example, the cyclooxygenase-2 selective is administered during a continuous period beginning on the same day as the beginning of the sodium ion channel blocker and extending to a period after the end of the sodium ion channel blocker. Alternatively, the cyclooxygenase-2 selective inhibitor and sodium ion channel blocker may be administered sequentially, meaning that they are administered at separate times during separate treatments. In one embodiment, for example, the cyclooxygenase-2 selective inhibitor is administered during a continuous period beginning prior to administration of the sodium ion channel blocker and ending after administration of the sodium ion channel blocker. Of course, it is also possible that the cyclooxygenase-2 selective inhibitor may be administered either more or less frequently than the sodium ion channel blocker. Moreover, it will be apparent to those skilled in the art that it is possible, and perhaps desirable, to combine various times and methods of administration in the practice of the present invention.

Combination Therapies

Generally speaking, it is contemplated that the composition employed in the practice of the invention may include one or more of any of the cyclooxygenase-2 selective inhibitors detailed above in combination with one or more of any of the sodium ion channel blockers detailed above. By way of a non-limiting example, Table 5a details a number of suitable combinations that are useful in the methods and compositions of the current invention. The combination may also include an isomer, a pharmaceutically acceptable salt, ester, or prodrug of any of the cyclooxygenase-2 selective inhibitors or sodium ion channel blockers listed in Table 5a.

TABLE 5A

| Cyclooxygenase-2 Selective | Sodium Ion |
|---|---|
| Inhibitor | Channel Blocker |
| a compound having formula I | disopyramide |
| a compound having formula I | procainimide |
| a compound having formula I | quinidine |
| a compound having formula I | tocainide |
| a compound having formula I | mexiletene |
| a compound having formula I | lidocane |
| a compound having formula I | phenytoin |
| a compound having formula I | fosphenytoin |
| a compound having formula I | flecainide |
| a compound having formula I | propafenone |
| a compound having formula I | morcizine |
| a compound having formula II | disopyramide |
| a compound having formula II | procainimide |
| a compound having formula II | quinidine |
| a compound having formula II | tocainide |
| a compound having formula II | mexiletene |
| a compound having formula II | lidocane |
| a compound having formula II | phenytoin |
| a compound having formula II | fosphenytoin |
| a compound having formula II | flecainide |
| a compound having formula II | propafenone |
| a compound having formula II | morcizine |
| a compound having formula III | disopyramide |
| a compound having formula III | procainimide |
| a compound having formula III | quinidine |
| a compound having formula III | tocainide |
| a compound having formula III | mexiletene |
| a compound having formula III | lidocane |
| a compound having formula III | phenytoin |
| a compound having formula III | fosphenytoin |
| a compound having formula III | flecainide |
| a compound having formula III | propafenone |
| a compound having formula III | morcizine |
| a compound having formula IV | disopyramide |
| a compound having formula IV | procainimide |
| a compound having formula IV | quinidine |
| a compound having formula IV | tocainide |
| a compound having formula IV | mexiletene |
| a compound having formula IV | lidocane |
| a compound having formula IV | phenytoin |
| a compound having formula IV | fosphenytoin |
| a compound having formula IV | flecainide |
| a compound having formula IV | propafenone |
| a compound having formula IV | morcizine |
| a compound having formula V | disopyramide |
| a compound having formula V | procainimide |
| a compound having formula V | quinidine |
| a compound having formula V | tocainide |
| a compound having formula V | mexiletene |
| a compound having formula V | lidocane |
| a compound having formula V | phenytoin |
| a compound having formula V | fosphenytoin |
| a compound having formula V | flecainide |
| a compound having formula V | propafenone |
| a compound having formula V | morcizine |

By way of further example, Table 5b details a number of suitable combinations that may be employed in the methods and compositions of the present invention. The combination may also include an isomer, a pharmaceutically acceptable salt, ester, or prodrug of any of the cyclooxygenase-2 selective inhibitors or sodium ion channel blockers listed in Table 5b.

TABLE 5b

| | Sodium |
|---|---|
| | Ion Channel |
| Cyclooxygenase-2 Selective Inhibitor | Blocker |
| a compound selected from the group consisting | disopyramide |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | procainimide |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | quinidine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | tocainide |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | mexiletene |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | lidocane |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | phenytoin |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | fosphenytoin |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | flecainide |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | propafenone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | morcizine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |

By way of yet further example, Table 5c details additional suitable combinations that may be employed in the methods and compositions of the current invention. The combination may also include an isomer, a pharmaceutically acceptable salt, ester, or prodrug of any of the cyclooxygenase-2 selective inhibitors or sodium ion channel blockers listed in Table 5c.

TABLE 5c

| Cyclooxygenase-2 | Sodium Ion |
|---|---|
| Selective Inhibitor | Channel Blocker |
| celecoxib | disopyramide |
| celecoxib | procainimide |
| celecoxib | quinidine |
| celecoxib | tocainide |
| celecoxib | mexiletene |
| celecoxib | lidocane |
| celecoxib | phenytoin |
| celecoxib | fosphenytoin |
| celecoxib | flecainide |
| celecoxib | propafenone |
| celecoxib | morcizine |
| deracoxib | disopyramide |
| deracoxib | procainimide |
| deracoxib | quinidine |
| deracoxib | tocainide |
| deracoxib | mexiletene |
| deracoxib | lidocane |
| deracoxib | phenytoin |
| deracoxib | fosphenytoin |
| deracoxib | flecainide |
| deracoxib | propafenone |
| deracoxib | morcizine |
| valdecoxib | disopyramide |
| valdecoxib | procainimide |
| valdecoxib | quinidine |
| valdecoxib | tocainide |
| valdecoxib | mexiletene |
| valdecoxib | lidocane |
| valdecoxib | phenytoin |
| valdecoxib | fosphenytoin |
| valdecoxib | flecainide |
| valdecoxib | propafenone |
| valdecoxib | morcizine |
| rofecoxib | disopyramide |
| rofecoxib | procainimide |
| rofecoxib | quinidine |
| rofecoxib | tocainide |
| rofecoxib | mexiletene |
| rofecoxib | lidocane |
| rofecoxib | phenytoin |
| rofecoxib | fosphenytoin |
| rofecoxib | flecainide |
| rofecoxib | propafenone |
| rofecoxib | morcizine |
| etoricoxib | disopyramide |
| etoricoxib | procainimide |
| etoricoxib | quinidine |
| etoricoxib | tocainide |
| etoricoxib | mexiletene |
| etoricoxib | lidocane |
| etoricoxib | phenytoin |
| etoricoxib | fosphenytoin |
| etoricoxib | flecainide |
| etoricoxib | propafenone |
| etoricoxib | morcizine |
| meloxicam | disopyramide |
| meloxicam | procainimide |
| meloxicam | quinidine |
| meloxicam | tocainide |
| meloxicam | mexiletene |
| meloxicam | lidocane |
| meloxicam | phenytoin |
| meloxicam | fosphenytoin |
| meloxicam | flecainide |
| meloxicam | propafenone |
| meloxicam | morcizine |
| parecoxib | disopyramide |
| parecoxib | procainimide |
| parecoxib | quinidine |
| parecoxib | tocainide |
| parecoxib | mexiletene |
| parecoxib | lidocane |
| parecoxib | phenytoin |
| parecoxib | fosphenytoin |
| parecoxib | flecainide |
| parecoxib | propafenone |
| parecoxib | morcizine |
| 4-(4-cyclohexyl-2-methyloxazol-5-yl)-2- | disopyramide |
| fluorobenzenesulfonamide | |
| 4-(4-cyclohexyl-2-methyloxazol-5-yl)-2- | procainimide |
| fluorobenzenesulfonamide | |
| 4-(4-cyclohexyl-2-methyloxazol-5-yl)-2- | quinidine |
| fluorobenzenesulfonamide | |
| 4-(4-cyclohexyl-2-methyloxazol-5-yl)-2- | tocainide |
| fluorobenzenesulfonamide | |
| 4-(4-cyclohexyl-2-methyloxazol-5-yl)-2- | mexiletene |
| fluorobenzenesulfonamide | |
| 4-(4-cyclohexyl-2-methyloxazol-5-yl)-2- | lidocane |
| fluorobenzenesulfonamide | |
| 4-(4-cyclohexyl-2-methyloxazol-5-yl)-2- | phenytoin |
| fluorobenzenesulfonamide | |
| 4-(4-cyclohexyl-2-methyloxazol-5-yl)-2- | fosphenytoin |
| fluorobenzenesulfonamide | |
| 4-(4-cyclohexyl-2-methyloxazol-5-yl)-2- | flecainide |
| fluorobenzenesulfonamide | |
| 4-(4-cyclohexyl-2-methyloxazol-5-yl )-2- | propafenone |
| fluorobenzenesulfonamide | |
| 4-(4-cyclohexyl-2-methyloxazol-5-yl )-2- | morcizine |
| fluorobenzenesulfonamide | |
| 2-(3,5-difluorophenyl)-3-(4- | disopyramide |
| (methylsulfonyl)phenyl)-2-cyclopenten-1- | |
| one | |
| 2-(3,5-difluorophenyl)-3-(4- | procainimide |
| (methylsulfonyl)phenyl)-2-cyclopenten-1- | |
| one | |
| 2-(3,5-difluorophenyl)-3-(4- | quinidine |
| (methylsulfonyl)phenyl)-2-cyclopenten-1- | |
| one | |
| 2-(3,5-difluorophenyl)-3-(4- | tocainide |
| (methylsulfonyl)phenyl)-2-cyclopenten-1- | |
| one | |
| 2-(3,5-difluorophenyl)-3-(4- | mexiletene |
| (methylsulfonyl)phenyl)-2-cyclopenten-1- | |
| one | |
| 2-(3,5-difluorophenyl)-3-(4- | lidocane |
| (methylsulfonyl)phenyl)-2-cyclopenten-1- | |
| one | |
| 2-(3,5-difluorophenyl)-3-(4- | phenytoin |
| (methylsulfonyl)phenyl)-2-cyclopenten-1- | |
| one | |
| 2-(3,5-difluorophenyl)-3-(4- | fosphenytoin |
| (methylsulfonyl)phenyl)-2-cyclopenten-1- | |
| one | |
| 2-(3,5-difluorophenyl)-3-(4- | flecainide |
| (methylsulfonyl)phenyl)-2-cyclopenten-1- | |
| one | |
| 2-(3,5-difluorophenyl)-3-(4- | propafenone |
| (methylsulfonyl)phenyl)-2-cyclopenten-1- | |
| one | |
| 2-(3,5-difluorophenyl)-3-(4- | morcizine |
| (methylsulfonyl)phenyl)-2-cyclopenten-1- | |
| one | |
| N-[2-(cyclohexyloxy)-4- | disopyramide |
| nitrophenyl]methanesulfonamide | |
| N-[2-(cyclohexyloxy)-4- | procainimide |
| nitrophenyl]methanesulfonamide | |
| N-[2-(cyclohexyloxy)-4- | quinidine |
| nitrophenyl]methanesulfonamide | |
| N-[2-(cyclohexyloxy)-4- | tocainide |
| nitrophenyl]methanesulfonamide | |
| N-[2-(cyclohexyloxy)-4- | mexiletene |
| nitrophenyl]methanesulfonamide | |
| N-[2-(cyclohexyloxy)-4- | lidocane |
| nitrophenyl]methanesulfonamide | |
| N-[2-(cyclohexyloxy)-4- | phenytoin |
| nitrophenyl]methanesulfonamide | |
| N-[2-(cyclohexyloxy)-4- | fosphenytoin |
| nitrophenyl]methanesulfonamide | |
| N-[2-(cyclohexyloxy)-4- | flecainide |
| nitrophenyl]methanesulfonamide | |
| N-[2-(cyclohexyloxy)-4- | propafenone |
| nitrophenyl]methanesulfonamide | |
| N-[2-(cyclohexyloxy)-4- | morcizine |
| nitrophenyl]methanesulfonamide | |
| 2-(3,4-difluorophenyl)-4-(3-hydroxy-3- | disopyramide |
| methylbutoxy)-5-[4- | |
| (methylsulfonyl)phenyl]-3(2H)- | |
| pyridazinone | |
| 2-(3,4-difluorophenyl)-4-(3-hydroxy-3- | procainimide |
| methylbutoxy)-5-[4- | |
| (methylsulfonyl)phenyl]-3(2H)- | |
| pyridazinone | |
| 2-(3,4-difluorophenyl)-4-(3-hydroxy-3- | quinidine |
| methylbutoxy)-5-[4- | |
| (methylsulfonyl)phenyl]-3(2H)- | |
| pyridazinone | |
| 2-(3,4-difluorophenyl)-4-(3-hydroxy-3- | tocainide |
| methylbutoxy)-5-[4- | |
| (methylsulfonyl)phenyl]-3(2H)- | |
| pyridazinone | |
| 2-(3,4-difluorophenyl)-4-(3-hydroxy-3- | mexiletene |
| methylbutoxy)-5-[4- | |
| (methylsulfonyl)phenyl]-3(2H)- | |
| pyridazinone | |
| 2-(3,4-difluorophenyl)-4-(3-hydroxy-3- | lidocane |
| methylbutoxy)-5-[4- | |
| (methylsulfonyl)phenyl]-3(2H)- | |
| pyridazinone | |
| 2-(3,4-difluorophenyl)-4-(3-hydroxy-3- | phenytoin |
| methylbutoxy)-5-[4- | |
| (methylsulfonyl)phenyl]-3(2H)- | |
| pyridazinone | |
| 2-(3,4-difluorophenyl)-4-(3-hydroxy-3- | fosphenytoin |
| methylbutoxy)-5-[4- | |
| (methylsulfonyl)phenyl]-3(2H)- | |
| pyridazinone | |
| 2-(3,4-difluorophenyl)-4-(3-hydroxy-3- | flecainide |
| methylbutoxy)-5-[4- | |
| (methylsulfonyl)phenyl]-3(2H)- | |
| pyridazinone | |
| 2-(3,4-difluorophenyl)-4-(3-hydroxy-3- | propafenone |
| methylbutoxy)-5-[4- | |
| (methylsulfonyl)phenyl]-3(2H)- | |
| pyridazinone | |
| 2-(3,4-difluorophenyl)-4-(3-hydroxy-3- | morcizine |
| methylbutoxy)-5-[4- | |
| (methylsulfonyl)phenyl]-3(2H)- | |
| pyridazinone | |
| 2-[(2,4-dichloro-6-methylphenyl)amino]-5- | disopyramide |
| ethyl-benzeneacetic acid | |
| 2-[(2,4-dichloro-6-methylphenyl)amino]-5- | procainimide |
| ethyl-benzeneacetic acid | |
| 2-[(2,4-dichloro-6-methylphenyl)amino]-5- | quinidine |
| ethyl-benzeneacetic acid | |
| 2-[(2,4-dichloro-6-methylphenyl)amino]-5- | tocainide |
| ethyl-benzeneacetic acid | |
| 2-[(2,4-dichloro-6-methylphenyl)amino]-5- | mexiletene |
| ethyl-benzeneacetic acid | |
| 2-[(2,4-dichloro-6-methylphenyl)amino]-5- | lidocane |
| ethyl-benzeneacetic acid | |
| 2-[(2,4-dichloro-6-methylphenyl)amino]-5- | phenytoin |
| ethyl-benzeneacetic acid | |
| 2-[(2,4-dichloro-6-methylphenyl)amino]-5- | fosphenytoin |
| ethyl-benzeneacetic acid | |
| 2-[(2,4-dichloro-6-methylphenyl)amino]-5- | flecainide |
| ethyl-benzeneacetic acid | |
| 2-[(2,4-dichloro-6-methylphenyl)amino]-5- | propafenone |
| ethyl-benzeneacetic acid | |
| 2-[(2,4-dichloro-6-methylphenyl)amino]-5- | morcizine |
| ethyl-benzeneacetic acid | |
| (3Z)-3-[(4-chlorophenyl)[4- | disopyramide |
| (methylsulfonyl)phenyl]methylene]dihydro- | |
| 2(3H)-furanone | |
| (3Z)-3-[(4-chlorophenyl)[4- | procainimide |
| (methylsulfonyl)phenyl]methylene]dihydro- | |
| 2(3H)-furanone | |
| (3Z)-3-[(4-chlorophenyl)[4- | quinidine |
| (methylsulfonyl)phenyl]methylene]dihydro- | |
| 2(3H)-furanone | |
| (3Z)-3-[(4-chlorophenyl)[4- | tocainide |
| (methylsulfonyl)phenyl]methylene]dihydro- | |
| 2(3H)-furanone | |
| (3Z)-3-[(4-chlorophenyl)[4- | mexiletene |
| (methylsulfonyl)phenyl]methylene]dihydro- | |
| 2(3H)-furanone | |
| (3Z)-3-[(4-chlorophenyl)[4- | lidocane |
| (methylsulfonyl)phenyl]methylene]dihydro- | |
| 2(3H)-furanone | |
| (3Z)-3-[(4-chlorophenyl)[4- | phenytoin |
| (methylsulfonyl)phenyl]methylene]dihydro- | |
| 2(3H)-furanone | |
| (3Z)-3-[(4-chlorophenyl)[4- | fosphenytoin |
| (methylsulfonyl)phenyl]methylene]dihydro- | |
| 2(3H)-furanone | |
| (3Z)-3-[(4-chlorophenyl)[4- | flecainide |
| (methylsulfonyl)phenyl]methylene]dihydro- | |
| 2(3H)-furanone | |
| (3Z)-3-[(4-chlorophenyl)[4- | propafenone |
| (methylsulfonyl)phenyl]methylene]dihydro- | |
| 2(3H)-furanone | |
| (3Z)-3-[(4-chlorophenyl)[4- | morcizine |
| (methylsulfonyl)phenyl]methylene]dihydro- | |
| 2(3H)-furanone | |
| (S)-6,8-dichloro-2-(trifluoromethyl)-2H-1- | disopyramide |
| benzopyran-3-carboxylic acid | |
| (S)-6,8-dichloro-2-(trifluoromethyl)-2H-1- | procainimide |
| benzopyran-3-carboxylic acid | |
| (S)-6,8-dichloro-2-(trifluoromethyl)-2H-1- | quinidine |
| benzopyran-3-carboxylic acid | |
| (S)-6,8-dichloro-2-(trifluoromethyl)-2H-1- | tocainide |
| benzopyran-3-carboxylic acid | |
| (S)-6,8-dichloro-2-(trifluoromethyl)-2H-1- | mexiletene |
| benzopyran-3-carboxylic acid | |
| (S)-6,8-dichloro-2-(trifluoromethyl)-2H-1- | lidocane |
| benzopyran-3-carboxylic acid | |
| (S)-6,8-dichloro-2-(trifluoromethyl)-2H-1- | phenytoin |
| benzopyran-3-carboxylic acid | |
| (S)-6,8-dichloro-2-(trifluoromethyl)-2H-1- | fosphenytoin |
| benzopyran-3-carboxylic acid | |
| (S)-6,8-dichloro-2-(trifluoromethyl)-2H-1- | flecainide |
| benzopyran-3-carboxylic acid | |
| (S)-6,8-dichloro-2-(trifluoromethyl)-2H-1- | propafenone |
| benzopyran-3-carboxylic acid | |
| (S)-6,8-dichloro-2-(trifluoromethyl)-2H-1- | morcizine |
| benzopyran-3-carboxylic acid | |
| lumiracoxib | disopyramide |
| lumiracoxib | procainimide |
| lumiracoxib | quinidine |
| lumiracoxib | tocainide |
| lumiracoxib | mexiletene |
| lumiracoxib | lidocane |
| lumiracoxib | phenytoin |
| lumiracoxib | fosphenytoin |
| lumiracoxib | flecainide |
| lumiracoxib | propafenone |
| lumiracoxib | morcizine |

Indications to be Treated

Generally speaking, the composition comprising a therapeutically effective amount of a cyclooxygenase-2 selective inhibitor and a therapeutically effective amount of a sodium ion channel blocker may be employed for symptomatic treatment of pain sensation and to treat inflammation, and inflammation mediated disorder.

One aspect of the invention encompasses administering the composition to a subject for symptomatic treatment of neuropathic pain. Neuropathic pain is pain that is due to functional abnormalities of the nervous system. In general, there are a variety of possible mechanisms by which nerve dysfunction can cause neuropathic pain: hyperactivity in primary afferent or central nervous system nociceptive neurons, loss of central inhibitory connections, and increased activity in sympathetic efferents. The composition of the invention may be utilized to treat neuropathic pain irrespective of the underlying mechanism causing the pain. Examples of causes of painful nerve injury that may be treated by the composition of the invention include accidental trauma, tumors, cerval or lumbar spine disease, and surgical procedures. Additionally, there are also toxic, metabolic, and hereditary causes of painful polyneuropathies, e.g., alcohol abuse, diabetes mellitus that may be treated by the composition of the invention.

In an alternative of this embodiment, the composition may be employed to treat allodynia and hyperalgesia neuropathic pain. Generally speaking, allodynia and hyperalgesia describes a particular type of pain sensation that differs from the customary perception of painful stimuli. Subjects who suffer from hyperalgesic pain feel painful stimuli more strongly than healthy subjects do. Alternatively, subjects who suffer from allodynia perceive stimuli that are not painful per se, such as contact or heat/cold, as pain.

Another aspect of the invention encompasses administering the composition to a subject for symptomatic treatment of nociceptive pain. Nociceptive pain includes all forms of somatic pain that result from damage or dysfunction of non-neural tissue. The composition may be employed to treat either acute or chronic nociceptive pain. Typically, acute nociceptive pain includes pain resulting from tissue-damaging stimulation such as that produced by injury or disease. Examples include postoperative pain, post traumatic pain, acute pancreatis, labor pain, muscle pain and pain accompanying myocardial infarction. Chronic nociceptive pain typically lasts for a longer duration of time relative to the duration of acute pain. Examples of chronic pain that may be treated by the composition include inflammatory pain; arthritis pain, cancer pain and other forms of persistent pain deriving from damaged or inflamed somatic tissue.

Yet another aspect of the invention encompasses administering the composition to lessen symptomatic pain resulting from a number of different disorders or disease states. In one embodiment, the composition may be administered to treat long-lasting allodynia resulting from herpes zoster (shingles) infection. In another embodiment, the composition may be administered to an AIDS patient, to treat pain in various stages of the disorder. In yet another embodiment, the composition may be administered to a subject with cancer to relieve pain resulting from either the cancer itself or for pain resulting from the treatment of cancer. By way of example, therapy with high doses of cytostatics for cancer generally causes pain. By way of further example, a tumor disorder itself can also elicit neuropathic pain that may be treated by the composition of the invention. In still another embodiment, a subject with chronic back pain, such as resulting from a compression of nerve roots of the spinal cord, can be treated by the composition of the invention. In yet another embodiment, a subject with a spinal cord injury, which often results in very severe pain sensations, may be treated by the composition of the invention.

A further aspect of the invention comprises administering the composition to treat inflammation or inflammation mediated disorders, such as those mediated by cyclooxygenase-2. Typical conditions benefited by cyclooxygenase-2 selective inhibition include the treatment or prevention of inflammation, and for treatment or prevention of other inflammation-associated disorders, such as, an analgesic in the treatment of pain and headaches, or as an antipyretic for the treatment of fever. For example, the composition is useful to treat or prevent arthritis, including but not limited to rheumatoid arthritis, spondyloarthopathies, gouty arthritis, osteoarthritis, systemic lupus erythematosus and juvenile arthritis. The composition is also useful in the treatment or prevention of asthma, bronchitis, menstrual cramps, tendinitis, bursitis, skin-related conditions such as psoriasis, eczema, burns and dermatitis, and from post-operative inflammation including ophthalmic surgery such as cataract surgery and refractive surgery. Moreover, the composition may be employed to treat or prevent gastrointestinal conditions such as inflammatory bowel disease, Crohn's disease, gastritis, irritable bowel syndrome and ulcerative colitis. The composition may also be employed in treating or preventing inflammation in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like.

### EXAMPLES

In the examples below, a combination therapy contains a sodium channel blocker and a Cox-2 selective inhibitor. The efficacy of such combination therapy can be evaluated in comparison to a control treatment such as a placebo treatment, administration of a Cox-2 inhibitor only, or administration of a sodium channel blocker only. By way of example, a combination therapy may contain lidocaine and celecoxib, quinidine and valdecoxib, procainamide and rofecoxib, or amiloride and celecoxib. It should be noted that these are only several examples, and that any of the sodium channel blockers and Cox-2 inhibitors of the present invention may be tested as a combination therapy. The dosages of a sodium channel blocker and Cox-2 inhibitor in a particular therapeutic combination may be readily determined by a skilled artisan conducting the study. The length of the study treatment will vary on a particular study and can also be determined by one of ordinary skill in the art. The sodium channel blocker and Cox-2 inhibitor can be administered by any route as described herein, but are preferably administered orally for human subjects.

### Example 1

### Evaluation of COX-1 and COX-2 Activity In Vitro

The COX-2 inhibitors suitable for use in this invention exhibit selective inhibition of COX-2 over COX-1 when tested in vitro according to the following activity assays.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 ug/ml.

The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

### Rat Carrageenan Foot Pad Edema Test

The anti-inflammatory properties of COX-2 selective inhibitors for use, along with their combination with a sodium channel blocker, in the present methods can be determined by the rat carrageenan footpad edema test. The carrageenan foot edema test is performed with materials, reagents and procedures essentially as described by Winter, et al., (Proc. Soc. Exp. Biol. Med., 111: 544, 1962). Male Sprague-Dawley rats are selected in each group so that the average body weight is as close as possible. Rats are fasted with free access to water for over sixteen hours prior to the test. The rats are dosed, e.g., orally (1 mL) with combination therapy suspended in vehicle containing 0.5% methylcellulose and 0.025% surfactant, or with placebo (e.g., vehicle alone). Alternative routes of administration, e.g., intraperitoneal, may also be used. One hour later, a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDs, in Non-steroidal Anti-Inflammatory Drugs, (J. Lombardino, ed. 1985)). The percentage inhibition indicates the efficacy of the combination therapy in comparison with placebo.

### Example 3

### Rat Plantar Test

The ability of COX-2 selective inhibitors along with sodium channel blockers for use in the method of the present invention to prevent hyperalgesia can be determined by the rat plantar test. The rat plantar test is performed with materials, reagents and procedures essentially as described by Hargreaves et al. (Pain. (1988) 32:77-88). Male Sprague-Dawley rats are selected in each group so that the average body weight is as close as possible. An inflammation is induced in the rats by intraplantar injection of an approximately 0.05% suspension of Mycobacterium butyricum. Six hours after this injection, a heat stimulus is applied by infrared ray onto the plantar face of the hind paw of the rat. The nociceptive reaction of the rat manifests itself by the withdrawal or the licking of the paw. The time of this pain reaction is then measured. Additionally the COX-2 selective inhibitor and sodium channel blocker are administered via, e.g., oral or intraperitoneal route approximately one hour before the plantar test. The average time of pain reaction in a group of drug-treated animals is then compared with that of a group of placebo-treated animals in order to determine the hyperalgesia preventative effect of the combination therapy of the present invention.

### Example 4

### Phenylbenzoquinone Test

The analgesic properties of COX-2 selective inhibitors along with sodium channel blockers for use in the present methods can be determined by the phenylbenzoquinone test. The phenylbenzoquinone test is performed with the materials, reagents, and procedures essentially as described in Siegmund et al. (Proc. Sec. Exp. Biol. Med. (1957) 95:729-731). Male Sprague-Dawley rats are selected in each group so that the average body weight is as close as possible. One hour after, e.g., the oral administration of the combination therapy or placebo, a 0.02% solution of phenylbenzoquinone is administered via the intra-peritoneal route to each rat. The number of pain reactions, measured as abdominal torsions and stretches, is then counted between the fifth and sixth minute after injection of the phenylbenzoquinone. The average number of pain reactions in a group of drug-treated animals is then compared with that of a group of placebo-treated animals in order to determine the analgesic properties of the composition of the present invention.

It should be noted that all of the above-mentioned procedures can be modified for a particular study, depending on factors such as a drug combination used, length of the study, subjects that are selected, etc. Such modifications can be designed by a skilled artisan without undue experimentation.

What is claimed is:

1. A method of treating pain, inflammation or an inflammation mediated disorder, the method comprising:
(a) diagnosing a subject in need of treatment for pain, inflammation or an inflammation mediated disorder; and
(b) administering to the subject a cyclooxygenase-2 selective inhibitor or an isomer, a pharmaceutically acceptable salt, ester, or prodrug thereof and a sodium ion channel blocker or an isomer, a pharmaceutically acceptable salt, ester, or prodrug thereof.

2. The method of claim 1 wherein the cyclooxgenase-2 selective inhibitor has a selectivity ratio of COX-1 IC₅₀ to COX-2 IC₅₀ not less than about 50.

3. The method of claim 1 wherein the cyclooxgenase-2 selective inhibitor has a selectivity ratio of COX-1 IC₅₀ to COX-2 IC₅₀ not less than about 100.

4. The method of claim 1 wherein the cyclooxygenase-2 selective inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl)-2H-1-benzopyran-3-carboxylic acid.

5. The method of claim 1 wherein the sodium ion channel blocker is selected from the group consisting of disopyramide, procainimide, quinidine, tocainide, mexiletene, lidocane, phenytoin, fosphenytoin, flecainide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encainide, and flunarizine or is an isomer, a pharmaceutically acceptable salt, ester, or prodrug thereof.

6. The method of claim 4 wherein the sodium ion channel blocker is selected from the group consisting of disopyramide, procainimide, quinidine, tocainide, mexiletene, lidocane, phenytoin, fosphenytoin, flecainide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encainide, and flunarizine or is an isomer, a pharmaceutically acceptable salt, ester, or prodrug thereof.

7. A method of treating pain, inflammation or an inflammation mediated disorder, the method comprising:
(a) diagnosing a subject in need of treatment for pain, inflammation or an inflammation mediated disorder; and
(b) administering to the subject a sodium ion channel blocker or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a chromene compound, the chromene compound comprising a benzothiopyran, a dihydroquinoline or a dihydronaphthalene.

8. The method of claim 7 wherein the cyclooxgenase-2 selective inhibitor has a selectivity ratio of COX-1 IC₅₀ to COX-2 IC₅₀ not less than about 50.

9. The method of claim 7 wherein the cyclooxgenase-2 selective inhibitor has a selectivity ratio of COX-1 IC₅₀ to COX-2 IC₅₀ not less than about 100.

10. The method of claim 7 wherein the cyclooxygenase-2 selective inhibitor is a compound having the formula 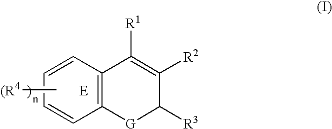
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴together with the carbon atoms to which it is attached and the remainder of ring E forms a naphthyl radical.

11. The method of claim 7 wherein the cyclooxgyenase-2 selective inhibitor is (S)-6,8-dichloro-2-(trifluoromethyl)-2H-1-benzopyran-3-carboxylic acid.

12. The method of claim 7 wherein the sodium ion channel blocker is selected from the group consisting of disopyramide, procainimide, quinidine, tocainide, mexiletene, lidocane, phenytoin, fosphenytoin, flecainide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encainide, and flunarizine or is an isomer, a pharmaceutically acceptable salt, ester, or prodrug thereof.

13. A method of treating pain, inflammation or an inflammation mediated disorder, the method comprising:
(a) diagnosing a subject in need of treatment for pain, inflammation or an inflammation mediated disorder; and
(b) administering to the subject a sodium ion channel blocker or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a tricyclic compound, the tricyclic compound containing a benzenesulfonamide or methylsulfonylbenzene moiety.

14. The method of claim 13 wherein the cyclooxgenase-2 selective inhibitor has a selectivity ratio of COX-1 IC₅₀ to COX-2 IC₅₀ not less than about 50.

15. The method of claim 13 wherein the cyclooxgenase-2 selective inhibitor has a selectivity ratio of COX-1 IC₅₀ to COX-2 IC₅₀ not less than about 100.

16. The method of claim 13 wherein the cyclooxygenase-2 selective inhibitor is a compound of the formula: 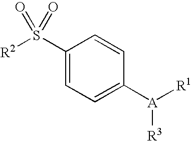
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

17. The method of claim 13 wherein the cyclooxygenase-2 selective inhibitor is selected from the group consisting of celecoxib, valdecoxib, parecoxib, deracoxib, rofecoxib, etoricoxib, and 2-(3,4-difluorophenyl)-4-(3-hydroxy-3-methyl butoxy)-5-[4-(methylsulfonyl)phenyl]-3(2H)-pyridazinone.

18. The method of claim 13 wherein the sodium ion channel blocker is selected from the group consisting of disopyramide, procainimide, quinidine, tocainide, mexiletene, lidocane, phenytoin, fosphenytoin, flecainide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encainide, and flunarizine or is an isomer, a pharmaceutically acceptable salt, ester, or prodrug thereof.

19. A method of treating pain, inflammation or an inflammation mediated disorder, the method comprising:
(a) diagnosing a subject in need of treatment for pain, inflammation or an inflammation mediated disorder; and
(b) administering to the subject a sodium ion channel blocker or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a phenyl acetic acid compound.

20. The method of claim 19 wherein the cyclooxgenase-2 selective inhibitor has a selectivity ratio of COX-1 IC₅₀ to COX-2 IC₅₀ not less than about 50.

21. The method of claim 19 wherein the cyclooxgenase-2 selective inhibitor has a selectivity ratio of COX-1 IC₅₀ to COX-2 IC₅₀ not less than about 100.

22. The method of claim 19 wherein the cyclooxygenase-2 selective inhibitor is a compound having the formula: 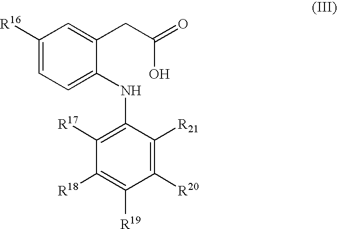
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl; and
provided that each of R¹⁷, R¹⁸, R¹⁹ and R²⁰ is not fluoro when R¹⁶ is ethyl and R¹⁹ is H.

23. The method of claim 22
wherein:
R¹⁶ is ethyl;
R¹⁷ and R¹⁹ are chloro;
R¹⁸ and R²⁰ are hydrogen; and
R²¹ is methyl.

24. The method of claim 19 wherein the sodium ion channel blocker is selected from the group consisting of disopyramide, procainimide, quinidine, tocainide, mexiletene, lidocane, phenytoin, fosphenytoin, flecainide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encainide, and flunarizine or is an isomer, a pharmaceutically acceptable salt, ester, or prodrug thereof.

25. A method of treating pain, inflammation or an inflammation mediated disorder, the method comprising:
(a) diagnosing a subject in need of treatment for pain, inflammation or an inflammation mediated disorder; and
(b) administering to the subject a cyclooxygenase-2 selective inhibitor selected from the group consisting of celecoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-(trifluoromethyl)-2H-1-benzopyran-3-carboxylic acid; and a sodium ion channel blocker selected from the group consisting of disopyramide, procainimide, quinidine, tocainide, mexiletene, lidocane, phenytoin, fosphenytoin, flecainide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encainide, and flunarizine or is an isomer, a pharmaceutically acceptable salt, ester, or prodrug thereof.

26. The method of claim 25 wherein the cyclooxygenase-2 selective inhibitor is celecoxib.

27. The method of claim 25 wherein the cyclooxygenase-2 selective inhibitor is deracoxib.

28. The method of claim 25 wherein the cyclooxygenase-2 selective inhibitor is valdecoxib.

29. The method of claim 25 wherein the cyclooxygenase-2 selective inhibitor is rofecoxib.

30. The method of claim 25 wherein the cyclooxygenase-2 selective inhibitor is etoricoxib.

31. The method of claim 25 wherein the cyclooxygenase-2 selective inhibitor is parecoxib.

32. The method of claim 25 wherein the cyclooxygenase-2 selective inhibitor is 2-(3,4-difluorophenyl)-4-(3-hydroxy-3-methylbutoxy)-5-[4-(methylsulfonyl)phenyl]-3(2H)-pyridazinone.

33. The method of claim 25 wherein the cyclooxygenase-2 selective inhibitor is (S)-6,8-dichloro-2-(trifluoromethyl)-2H-1-benzopyran-3-carboxylic acid.

34. The method of claim 25 wherein the cyclooxygenase-2 selective inhibitor is lumiracoxib.

35. The method of claim 1 wherein the inflammation mediated disorder is arthritis.

36. The method of claim 1 wherein the inflammation mediated disorder is pain.

37. The method of claim 1 wherein the inflammation mediated disorder is a gastrointestinal disorder.

38. The method of claim 37 wherein the gastrointestinal disorder is selected from the group consisting of inflammatory bowel disease, Crohn's disease, gastritis, irritable bowel syndrome and ulcerative colitis.
